(19) United States
(12) Patent Application Publication
(10) Pub. No.: US 2004/0055025 A1
(43) Pub. Date: Mar. 18, 2004

(54) Immune response replication in cloned animals

(75) Inventors: Erik J. Forsberg, Oregon, WI (US); Gregory H. Leno, Madison, WI (US); Jeffrey Betthauser, Windsor, WI (US); Kenneth Eilertsen, Waterlo, WI (US); Michael D. Bishop, Rio, WI (US)

Correspondence Address: FOLEY & LARDNER, P.O. BOX 80278, SAN DIEGO, CA, 92138-0278, US

(73) Assignee: Infigen, Inc.

(21) Appl. No.: 10/355,812

(22) Filed: Jan. 30, 2003

Related U.S. Application Data
(60) Provisional application No. 60/353,595, filed on Jan. 30, 2002.

(51) Int. Cl. A01K 67/027; C12P 21/04

(52) U.S. Cl. 800/14; 800/15; 800/16; 800/17; 435/70.21

(57) ABSTRACT

The present invention provides a method and materials for reproducing an immune response of a mammal against one or more antigens of interest. The method preferably involves cloning a founder mammal and producing an immune response in the clone that is substantially identical to the immune response of the founder animal to the antigen or antigens of interest. Accordingly, a source of valuable antibodies can be maintained despite the death or illness of the antibody producing animal.

### RELATED APPLICATION

This application claims priority to provisional U.S. Application No. 60/353,595 filed Jan. 30, 2002 which is hereby incorporated by reference in its entirety, including all tables, figures and claims.

### FIELD OF THE INVENTION

The present invention relates to methods and compositions for replicating a animal by nuclear transfer cloning, where an immune response in the cloned animal is substantially identical to that of the founder mammal used to establish the clone.

### BACKGROUND OF THE INVENTION

The following discussion of the background of the invention is merely provided to aid the reader in understanding the invention and is not admitted to describe or constitute prior art to the present invention.

Various animals, and in particular farm animals and livestock, have long been recognized as having economic value. This economic value derives from desirable characteristics or traits which are present in the animal. The economic value of specific individual animals, such as winning thoroughbreds and grand champion livestock, has climbed into the millions of dollars. Many of these animals have also commanded substantial amounts of money for breeding purposes so that progeny produced will have the desirable genetic characteristics of the parent animals.

With recent advances in biotechnology, genetic modification has provided many animals that have the potential of producing substances and pharmaceuticals worth ten to hundreds of millions of dollars. Transgenic animals have been produced to provide a source of many different therapeutic molecules, including antibodies, which are useful in the treatment, diagnosis and prevention of a wide spectrum of diseases, disorders and conditions. In many instances, only an individual or small number of animals are the exclusive source of therapeutic molecules. As such, the death or illness of this individual or these animals can wipe out the entire supply of a therapeutic protein. Cutting off the supply of therapeutics can have serious consequences for the owners of the animals, who derive economic advantage from them, not to mention people dependent upon these therapeutics for the diagnosis and treatment of medical conditions.

Unfortunately, many animals with desirable and economically valuable characteristics have, for various reasons, been unable to pass on their desirable characteristics to their offspring. Accordingly, attempts have been made to replicate the animal and thus maintain the trait which conferred value upon the animal, typically by cloning the animal. However, even minor modifications to a trait of value, such as protein or antibody production, in certain animals can significantly diminish their value.

Thus, there remains a need not only to provide animals which have similar traits as those recognized in specific animals, but also to substantially reproduce the valuable characteristics of certain animals. This is especially true where a substantial financial and scientific investment has been devoted to providing an animal with very desirable traits, such as is the case with transgenic animals.

### SUMMARY OF THE INVENTION

The present invention relates to methods and compositions for producing one or more mammals that exhibit an immune response that is substantially identical to an immune response exhibited by a founder mammal. By transferring cells of the immune system, including hematopoetic cells, for example by bone marrow or lymphatic cell transplantation, the skilled artisan can reproduce the immune response of the founder mammal in one or more recipient mammals. These methods are known in the art by the phrase “adoptive transfer.”

By combining adoptive transfer methods with nuclear transfer cloning methods, limitations related to graft rejection and graft-versus-host rejection can be avoided by providing one or more recipient mammals that are essentially genetically identical to the founder mammal. Such methods can be particularly advantageous for reproducing and/or expanding the number of mammals producing a valuable immune response, such as a polyclonal antibody exhibiting advantageous characteristics.

Thus, in a first aspect, the present invention relates to materials and methods for replicating an immune response to one or more antigens of interest exhibited by a first animal, preferably a mammal, in one or more second animals. In certain embodiments, an immune response is replicated by adoptive transfer methods.

The phrase “replicating an immune response” as used herein refers to methods for providing an immune response to one or more selected antigens in one or more second animals that is substantially identical to the immune response for the same antigen(s) in a first animal. Numerous methods are known to the skilled artisan for replicating an immune response to antigen(s) of interest. For example, in certain preferred embodiments, an immune response to such antigen(s) can be produced by subjecting the second animal(s) to the same immunization strategy as that of the first animal; the genetic elements responsible for the immune response to such antigen(s) can be placed into the animal by recombinant DNA methods; and/or one or more hematopoetic cells obtained from the first animal can be transferred to the second animal(s) by adoptive transfer methods.

The term “hematopoetic cells” as used herein refers to those cells that form the cells circulating in the blood, including precursor cells for red blood cells, lymphocytes, macrophages, monocytes, eosinophils, basophils, neutrophils, natural killer cells, and platelets. While immune system cells can be found in the blood, the skilled artisan will understand that cells of the immune system typically travel freely between the blood, tissues, and lymphatic system. Hematopoetic cells, and precursors of lymphocytes in particular, in mammals are typically found in the bone marrow, and may be transferred by procedures known in the art as “bone marrow transplantation.” Hematopoetic cells may also be found in, for example, the thymus.

The skilled artisan will understand that not all cells obtained from a source of hematopoetic cells need to be transferred from a donor to an acceptor in order to successfully transfer a functional hematopoetic system or an immune system that targets a specific antigen. For example, stem cells (e.g., hematopoetic stem cells, self-replicating stem cells, pluripotent stem cells) may be purified and transferred. Similarly, stem cells may be obtained from a stem cell culture and used to transfer a functional hematopoetic system. Purification in this context does not refer to removing all materials from the sample other than the analyte or cell of interest. Instead, purification refers to a procedure that enriches the amount of one or more analytes or cells of interest relative to one or more other components of a sample.

In certain embodiments, the immune response that is replicated can be a humoral immune response (e.g., an antibody-mediated immune response), and/or a cell-mediated immune response (e.g., a response in which antibody-producing cells play only a minor role). The skilled artisan will understand that, in order to replicate an immune response for a given antigen of interest, the entire immune repertoire of the founder mammal need not be replicated if replication of only a subset of the entire repertoire (e.g. a response to one or a few antigens) is desired.

The term “immunization” as used herein refers to methods known to the skilled artisan for inducing an immune response in an animal by introducing (e.g., by injection, by mucosal challenge, etc.) a preparation into the animal under conditions designed to stimulate an immune response. For example, an antigenic composition can be injected, with or without the use of adjuvants. See, e.g., Berggren-Thomas et al., J. Mammal Sci. 64: 1302-12 (1987); Gyorkos et al., Can J Public Health. 85 Suppl 1:S14-30 (1994). Alternatively, DNA preparations can be injected in a method known as “genetic immunization.” See, e.g., Davis et al., Biotechniques 21: 92-4, 96-9 (1996). See also, Hanley et al., “Review of Polyclonal Antibody Production Procedures in Mammals and Poultry,” ILAR Journal 37: 93-118 (1995). In preferred embodiments, such immunization methods can be combined. As used in the present invention, immunization includes, but does not require, providing complete immunity against an antigen of interest in an animal.

The phrase “recombinant DNA methods” as used herein in reference to replicating an immune response, refers to methods that transfer the DNA responsible for the immune response of interest into a recipient animal in a functional manner. The skilled artisan will understand that generation of a robust immune response requires the rearrangement of various gene segments, resulting in a mature immunoglobulin or immunoglobulin-related gene. Expression systems can be inserted into cells that permit the functional expression of immunoglobulin or immunoglobulin-related proteins from such genes. See, e.g., Boel and Verlaan, J. Immunol. Meth. 239: 153-66 (2000); Watkins and Ouwehand, Vox Sanguinis 78: 72-9 (2000); O'Brien et al., Proc. Natl. Acad. Sci. USA 96: 640-5 (1999); Li et al., J. Immunol. Meth. 236: 133-46 (2000).

The phrase “adoptive transfer” as used herein refers to methods for transferring cells of the immune response between animals. For example, hematopoetic cells can be transferred, preferably be performed by transferring hematopoetic stem cells from one animal to another, commonly referred to as “bone marrow transplantation.” See, e.g., Crombleholme et al., J. Ped. Surg. 25: 885-92 (1990); Zanjani et al., Blood Cells 17: 349-63 (1991); Jankowski and Ildstad, Hum. Immunol. 52: 155-61 (1997); Pu et al., Cell. Immunol. 198: 30-43 (1999). The skilled artisan will understand that, in an adoptive transfer procedure, an adult animal may serve as a donor of hematopoetic cells to either a fetal or a live-born recipient animal. Alternatively, immune cells obtained from another source (lymph nodes, thymus, etc.) can be transferred between animal.

Adoptive transfer of the one or more immune system cells of the founder animal can further comprise enriching the immune system cells from the founder animal by transferring specifically selected immune system cells of the founder animal which are responsible for the immune response to the antigen of interest and/or increasing the number of immune system cells harvested from the founder animal prior to transferring the immune system cells to the cloned animal. To ensure proper engraftment of the immune system cells of the founder animal to the immune system of the cloned animal(s), the immune system of the cloned animal(s) can be partially or fully ablated. After adoptive transfer of one or more cells of the immune system of the founder animal to the cloned animal(s), the cloned animal(s) can also be immunized with the antigen of interest to enhance the immune response.

The phrase “substantially identical” as used herein in reference to an immune response to an antigen, refers to comparing the immune response to the antigen in one animal to the immune response to the same antigen in a second animal, and determining that the immune responses are within a factor of 10, and preferably within a factor of two, of one another by one or more measures of immune response commonly used in the art. Humoral immune responses can be measured, for example, by determining an antibody titer. See, e.g., Vincent et al., J. Virol 75: 1516-21 (2001); van der Poel et al., Am. J. Vet. Res. 60: 1098-101 (1999); Hohdatsu et al., J. Vet. Med. Sci. 59: 377-81 (1997); Kodama et al., J. Clin. Microbiol. 35: 839-42 (1997). Similarly, cell-mediated immunity can be measured in lymphoproliferation or contact-sensitivity tests, or by measuring the production of one or more cytokines. See, e.g., Nuallain et al., Vet. Res. Commun. 21: 19-28 (1997); Borleffs et al., Scand. J. Immunol. 37: 634-6 (1993); Gupta et al., Indian J. Exp. Biol. 28: 1021-5 (1990).

Any animal can be used as the founder and/or recipient animals in the immunity transfer procedures described herein. For example, avian, and preferably agricultural poultry species such as chickens, cows, ducks, turkeys, etc., can be used. In particularly preferred embodiments, the founder and/or recipient animals are mammalian, and most preferably ungulates. Most preferably, the founder and recipient animals are of the same species, although cross-species transfer is within the scope of the invention.

The term “mammalian” as used herein refers to any mammal of the class Mammalia. Preferably, a mammal is a placental, a monotreme and a marsupial. Most preferably, a mammal is a canid, felid, murid, leporid, ursid, mustelid, ungulate, ovid, suid, equid, bovid, caprid, cervid, a non-human primate, and a human. The term “non-human mammal” refers to all mammals except humans.

The term “canid” as used herein refers to any mammal of the family Canidae. Preferably, a canid is a wolf, a jackal, a fox, and a domestic dog. The term “felid” as used herein refers to any mammal of the family Felidae. Preferably, a felid is a lion, a tiger, a leopard, a cheetah, a cougar, and a domestic cat. The term “murid” as used herein refers to any mammal of the family Muridae. Preferably, a murid is a mouse and a rat. The term “leporid” as used herein refers to any mammal of the family Leporidae. Preferably, a leporid is a rabbit. The term “ursid” as used herein refers to any mammal of the family Ursidae. Preferably, a ursid is a bear. The term “mustelid” as used herein refers to any mammal of the family Mustelidae. Preferably, a mustelid is a weasel, a ferret, an otter, a mink, and a skunk. The term “primate” as used herein refers to any mammal of the Primate order. Preferably, a primate is an ape, a monkey, a chimpanzee, and a lemur.

The term “ungulate” as used herein refers to any mammal of the polyphyletic group formerly known as the taxon Ungulata. Preferably, an ungulate is a camel, a hippopotamus, a horse, a tapir, and an elephant. Most preferably, an ungulate is a sheep, a cow, a goat, and a pig. Especially preferred in the bovine species are Bos taurus, Bos indicus, and Bos buffaloes cows or bulls. The term “ovid” as used herein refers to any mammal of the family Ovidae. Preferably, an ovid is a sheep. The term “suid” as used herein refers to any mammal of the family Suidae. Preferably, a suid is a pig or a boar. The term “equid” as used herein refers to any mammal of the family Equidae. Preferably, an equid is a zebra or an ass. Most preferably, an equid is a horse. The term “bovid” as used herein refers to any mammal of the family Bovidae. Preferably, an bovid is an antelope, an oxen, a cow, and a bison. The term “caprid” as used herein refers to any mammal of the family Caprinae. Preferably, a caprid is a goat. The term “cervid” as used herein refers to any mammal of the family Cervidae. Preferably, a cervid is a deer.

The term “live born” as used herein preferably refers to an animal that exists ex utero. A “live born” animal may be an animal that is alive for at least one second from the time it exits the maternal host. A “live born” animal may not require the circulatory system of an in utero environment for survival. A “live born” animal may be an ambulatory animal. Such animals can include pre- and post-pubertal animals. A live born animal may lack a portion of what exists in a normal animal of its kind.

The adoptive transfer methods described herein can be of limited usefulness for replicating an immune response in allogenic animal, due to the limitations imposed by graft failure resulting from rejection by the recipient, and by graft-versus-host disease resulting from attack of the host by the transferred immune cells. Such difficulties can be overcome by the use of immunosupressive agents; however, the use of such agents are associated with significant morbidity. Thus, in another aspect, the present invention relates to methods and compositions for replicating an immune response exhibited by a first animal in one or more second animals that are clones of the first animal. Such cloned animals are essentially autologous with respect to the immune system of the founder animal used to establish the clones.

These techniques include providing one or more second animals that are clones of a first, founder animal, the cloned animal(s) being produced through nuclear transfer cloning methods, and subsequently producing an immune response to an antigen of interest in the cloned animal(s) that is substantially identical to the immune response of the founder animal to the same antigen by one or more of the methods disclosed herein.

The term “nuclear transfer” as used herein refers to introducing a full complement of nuclear DNA from one cell to an enucleated cell. Nuclear transfer methods are well known to a person of ordinary skill in the art. See., e.g., U.S. Pat. Nos. 6,258,998, 6,011,197, 6,107,543, and 5,945,577; U.S. Provisional Patent Application No. 60/221,434, filed Jul. 28, 2000, entitled “Method of Cloning Porcine Animals; Nagashima et al., 1997, Mol. Reprod. Dev. 48: 339-343; Nagashima et al., 1992, J. Reprod. Dev. 38: 73-78; Prather et al., 1989, Biol. Reprod. 41: 414-419; Prather et al., 1990, Exp. Zool. 255: 355-358; Saito et al., 1992, Assis. Reprod. Tech. Andro. 259: 257-266; and Terlouw et al., 1992, Theriogenology 37: 309, each of which is incorporated herein by reference in its entirety including all figures, tables and drawings.

As discussed above, an immune response can be replicated by placing the genetic elements responsible for the immune response to such antigen(s) into an animal by recombinant DNA methods. In certain embodiments, this may be accomplished by preparing a cloned animal using a transgenic cell comprising one or more copies of the appropriate immunoglobulin or immunoglobulin-related genes as a nuclear donor in a nuclear transfer procedure.

Methods and tools for insertion, deletion, and mutation of nuclear DNA of mammalian cells are well-known to a person of ordinary skill in the art. See, Molecular Cloning, a Laboratory Manual, 2nd Ed., 1989, Sambrook, Fritsch, and Maniatis, Cold Spring Harbor Laboratory Press; U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al., issued Mar. 18, 1997; and PCT publication WO 93/22432, “Method for Identifying Transgenic Pre-Implantation Embryos”; WO 98/16630, Piedrahita & Bazer, published Apr. 23, 1998, “Methods for the Generation of Primordial Germ Cells and Transgenic Mammal Species,” each of which is incorporated herein by reference in its entirety, including all figures, drawings, and tables. These methods include techniques for transfecting cells with foreign DNA fragments designed such that they effect replacement, insertion, deletion, and/or mutation of the target DNA genome.

The terms “transfected” and “transfection” as used herein refer to methods of delivering exogenous DNA into a cell. These methods involve a variety of techniques, such as treating cells with calcium phosphate, an electric field, liposomes, polycationic micelles, or detergent, which induce cells to take up, or render a host cell outer membrane or wall permeable to, nucleic acid molecules of interest. These specified methods are not limiting and the invention relates to any transfection technique well known to a person of ordinary skill in the art. See, e.g., Molecular Cloning, a Laboratory Manual, 2nd Ed., 1989, Sambrook, Fritsch, and Maniatis, Cold Spring Harbor Laboratory Press and Transgenic Mammals, Generation and Use, 1997, Edited by L. M. Houdebine, Hardwood Academic Publishers, Australia, both of which were previously incorporated by reference.

The term “regulatory element” as used herein refers to a DNA sequence that can increase or decrease an amount of product produced from another DNA sequence. A regulatory element can cause the constitutive production of the product (e.g., the product can be expressed constantly). Alternatively, a regulatory element can enhance or diminish production of a recombinant product in an inducible fashion (e.g., the product can be expressed in response to a specific signal). A regulatory element can be controlled, for example, by nutrition, by light, or by adding a substance to the transgenic organism's system. Examples of regulatory elements well-known to those of ordinary skill in the art are promoters, enhancers, insulators, and repressors. See, e.g., Transgenic Mammals, Generation and Use, 1997, Edited by L. M. Houdebine, Hardwood Academic Publishers, Australia, hereby incorporated herein by reference in its entirety including all figures, tables, and drawings.

The term “promoters” or “promoter” as used herein refers to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism species can be utilized to enhance product expression from a DNA sequence that originates from another organism species. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art. Examples of promoter elements are described hereafter.

The term “enhancers” or “enhancer” as used herein refers to a DNA sequence that is located adjacent to the DNA sequence that encodes a recombinant product. Enhancer elements are typically located upstream of a promoter element or can be located downstream of a coding DNA sequence (e.g., a DNA sequence transcribed or translated into a recombinant product or products). Hence, an enhancer element can be located 100 base pairs, 200 base pairs, or 300 or more base pairs upstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

The term “insulators” or “insulator” as used herein refers to DNA sequences that flank the DNA sequence encoding the recombinant product. Insulator elements can direct recombinant product expression to specific tissues in an organism. Multiple insulator elements are well known to persons of ordinary skill in the art. See, e.g., Geyer, 1997, Curr. Opin. Genet. Dev. 7: 242-248, hereby incorporated herein by reference in its entirety, including all figures, tables, and drawings.

The term “repressor” or “repressor element” as used herein refers to a DNA sequence located in proximity to the DNA sequence that encodes recombinant product, where a repressor sequence can decrease an amount of recombinant product expressed from that DNA sequence. Repressor elements can be controlled by binding of a specific molecule or specific molecules to a repressor element DNA sequence. These molecules can either activate or deactivate a repressor element. Multiple repressor elements are available to a person of ordinary skill in the art.

As discussed herein, the methods and compositions of the present invention can be particularly useful in replicating the immune response of an animal which exhibits a valuable immune response. In yet another aspect, then, the invention features a method of using an animal having a replicated immune response, comprising the step of isolating and/or purifying at least one component from the animal. In preferred emnodiments, the isolated component is an antibody, most preferably a polyclonal antibody.

The term “component” as used herein can relate to any portion of an animal. A component can be selected from the group consisting of fluid, biological fluid, cell, tissue, organ, gamete, embryo, and fetus. For example, precursor cells, as defined previously, may arise from fluids, biological fluids, cells, tissues, organs, gametes, embryos, and fetuses isolated from cloned organisms of the invention.

The term “purification” as used herein refers to increasing the specific activity of a particular polypeptide or polypeptides in a sample. Specific activity can be expressed as a ratio between the activity or amount of a target polypeptide and the concentration of total polypeptide in the sample. Activity can be catalytic activity and/or binding activity, for example. Also, specific activity can be expressed as a ratio between the concentration of target polypeptide and the concentration of total polypeptide. Purification methods include dialysis, centrifugation, and column chromatography techniques, which are well-known procedures to a person of ordinary skill in the art. See, e.g., Young et al., 1997, “Production of biopharmaceutical proteins in the milk of transgenic dairy mammals,” BioPharm 10(6): 34-38.

Another aspect of the present invention provides one or more second embryos, fetuses, or live-born animals that have an immune system exhibiting an immune response to at least one antigen of interest that is substantially the same as the immune response of a first animal, or one or more embryos, fetuses, or live-born animals that derive from such a second animal embryo, fetus, or live-born animal by cloning or by reproduction.

The term “enriched” means both purifying in an numerical sense and purifying in a functional sense. “Enriched” does not imply that there are no undesired cells are present, just that the relative amount of the cells of interest have been significantly increased in either a numeric or functional sense.

As used herein, “cell”, “cell line”, and “cell culture” may be used interchangeably and all such designations include progeny. It is also understood that all progeny may not be precisely identical in DNA content, due to deliberate or inadvertent mutations.

In yet another aspect, the present invention relates to improved methods and compositions for preparing cloned animals by nuclear transfer. In particular, cells to be used as a source of nuclear donor material are contacted with one or more compounds that affect cholesterol biosynthesis prior to the use of the cell, or its nucleus, as a nuclear donor. In certain embodiments, these compounds can be inhibitors of one or more enzymes in the cellular cholesterol biosynthesis pathway. Such compounds can advantageously increase the efficiency of nuclear transfer methods by increasing the rate at which cloned embryos, fetuses, and/or animals are produced.

There are numerous enzymes known to be involved in the biosynthesis of cholesterol, such as hydroxymethylglutaryl-CoA synthase (HMG-CoA synthase), HMG-CoA reductase, mevalonate kinase, phosphomevalonate kinase, pyrophosphomevalonate decarboxylase, isopentenyl pyrophosphate isomerase, dimethylallyl transferase, presqualene synthase, squalene synthase, squalene monooxidase, and squalene epoxide lanosterol cyclase. Similarly, there are numerous intermediate products involved in cholesterol biosynthesis, such as acetyl-CoA, acetoacetyl-CoA, hydroxymethylglutaryl-CoA (HMG-CoA), L-mevalonic acid, 5-phosphomevalonic acid, 5-pyrophosphomevalonic acid, 3-isopentenylypyrophosphoric acid, 3,3-dimethylallylpyrophosphoric acid, isopentenyl pyrophosphate, geranyl pyrophosphoric acid, famesyl pyrophosphoric acid, presqualene pyrophosphate, squalene, squalene 2,3 epoxide, and lanosterol. Thus, the term “compound that affect cholesterol biosynthesis” as used herein refers to those compounds that exert a direct action on one of these enzymes or intermediate products. Such a direct action can be, for example, to inhibit an enzyme, alter the Kₘ or Vₘₐₓ of an enzyme, or increase or decrease the intracellular concentration of an intermediate.

The term “inhibitor of an enzyme in the cholesterol biosynthesis pathway” as used herein refers to a compound that reduces the rate or amount of product produced by at least one enzyme listed above under physiological conditions. While the actions of such an inhibitor on a cell to be used as a source of nuclear donor material are believed to be mediated by inhibition of an enzyme in the cholesterol biosynthesis pathway, this term is not intended to require that such inhibition be actually demonstrated within the nuclear donor cell. Rather, the term refers to the fact that such a cell contains an enzyme that can be inhibited by the compound.

In preferred embodiments, an inhibitor of an enzyme in the cholesterol biosynthesis pathway is an inhibitor of HMG-CoA reductase. Such compounds, some of which may be referred to as “statins,” have been shown to regulate a key early step in cholesterol biosynthesis. Numerous inhibitors of HMG-CoA reductase, such as lovastatin, simvistatin, pravastatin, fluvastatin, atorvastatin, and cerivastatin, and methods of producing such compounds, are known in the art. See, e.g., U.S. Pat. Nos. 4,582,914; 4,611,067; 4,665,091; 4,668,699; 4,738,982; 4,795,811; 4,851,436; 4,857,546; 4,873,345; 4,997,775; 5,021,453; 5,032,602; 5,064,841; 5,075,311; 5,081,136; 5,098,931; 5,102,911; 5,116,870; 5,134,155; 5,145,857; 5,202,327; 5,250,561; 5,256,692; 5,369,123; 5,385,932; 6,043,064; and 6,268,186, each of which is hereby incorporated by reference in its entirety.

Thus, in various preferred embodiments, a cell to be used as a source of nuclear donor material is contacted with one or more inhibitors of HMG-CoA reductase. Such contacting can be, for example, by incubating the cell in a medium comprising one or more inhibitors of HMG-CoA reductase. The cell can be contacted for various lengths of time from about 1 minute to about 240 hours, preferably from about 10 minutes to about 120 hours, more preferably from about 30 minutes to about 96 hours, even more preferably from about 2 hours to about 72 hours, and most preferably from about 12 hours to about 48 hours. The term “about” in this context refers to +/−10% of the time in question. Thus, a cell contacted for a preferred time of “about 24 hours” refers to 21.6-26.4 hours.

Preferably, the cell to be used as a source of nuclear donor material is a mammalian cell. In preferred embodiments, (1) the mammalian cell is an ungulate cell; (2) the ungulate is selected from the group consisting of bovids, ovids, cervids, suids, equids and camelids; (3) the ungulate is bovine; (4) the mammalian cell is a nonembryonic cell; (5) the mammalian cell is a fetal cell; and (6) the mammalian cell is an adult cell.

In certain embodiments, the cell to be used as a source of nuclear donor material is a cell obtained from a primary culture. The terms “primary culture” and “primary cell” refer to cells taken from a tissue source, and their progeny, grown in culture before subdivision and transfer to a subculture.

The term “cultured” as used herein in reference to cells refers to one or more cells that are undergoing cell division or not undergoing cell division in an in vitro environment. An in vitro environment can be any medium known in the art that is suitable for maintaining cells in vitro, such as suitable liquid media or agar, for example. Specific examples of suitable in vitro environments for cell cultures are described in Culture of Animal Cells: a manual of basic techniques (3^{rd} edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. MorganJohn Wiley and Sons, Ltd., each of which is incorporated herein by reference in its entirety including all figures, tables, and drawings. Cells may be cultured in suspension and/or in monolayers with one or more substantially similar cells. Cells may be cultured in suspension and/or in monolayers with a heterogeneous population of cells. The term “heterogeneous” as utilized in the previous sentence can relate to any cell characteristics, such as cell type and cell cycle stage, for example. Cells may be cultured in suspension, cultured as monolayers attached to a solid support, and/or cultured on a layer of feeder cells, for example. The term “feeder cells” is defined hereafter. Furthermore, cells may be successfully cultured by plating the cells in conditions where they lack cell to cell contact. Preferably, cultured cells undergo cell division and are cultured for at least 5 days, more preferably for at least 10 days or 20 days, and most preferably for at least 30 days. Preferably, a significant number of cultured cells do not terminate while in culture. The terms “terminate” and “significant number” are defined hereafter. Nearly any type of cell can be placed in cell culture conditions. Cultured cells can be utilized to establish a cell line.

The terms “plated” or “plating” as used herein in reference to cells refer to establishing cell cultures in vitro. For example, cells can be diluted in cell culture media and then added to a cell culture plate or cell culture dish. Cell culture plates are commonly known to a person of ordinary skill in the art. Cells may be plated at a variety of concentrations and/or cell densities. In preferred embodiments, plated cells may grow to confluence.

The meaning of the term “cell plating” can also extend to the term “cell passaging.” Cells of the invention can be passaged using cell culture techniques well known to those skilled in the art. The term “cell passaging” refers to such techniques which typically involve the steps of (1) releasing cells from a solid support and disassociation of these cells, and (2) diluting the cells in fresh media suitable for cell proliferation. Cells can be successfully grown by plating the cells in conditions where they lack cell to cell contact. Cell passaging may also refer to removing a portion of liquid medium bathing cultured cells and adding liquid medium from another source to the cell culture to dilute the cell concentration.

The term “proliferation” as used herein in reference to cells refers to a group of cells that can increase in size and/or can increase in numbers over a period of time.

In certain embodiments, the cell to be used as a source of nuclear donor material is obtained from a confluent culture, a suspension culture, and/or a culture that is not serum-starved.

The term “confluence” as used herein refers to a group of cells where a large percentage of the cells are physically contacted with at least one other cell in that group. Confluence may also be defined as a group of cells that grow to a maximum cell density in the conditions provided. For example, if a group of cells can proliferate in a monolayer and they are placed in a culture vessel in a suitable growth medium, they are confluent when the monolayer has spread across a significant surface area of the culture vessel. The surface area covered by the cells preferably represents about 50% of the total surface area, more preferably represents about 70% of the total surface area, and most preferably represents about 90% of the total surface area.

The term “suspension” as used herein refers to cell culture conditions in which the cells are not attached to a solid support. Cells proliferating in suspension can be stirred while proliferating using apparatus well known to those skilled in the art.

The term “monolayer” as used herein refers to cells that are attached to a solid support while proliferating in suitable culture conditions. A small portion of the cells proliferating in the monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “substantially similar” as used herein in reference to mammalian cells refers to cells from the same organism and the same tissue. Substantially similar can also refer to cell populations that have not significantly differentiated. For example, preferably less than 15% of the cells in a population of cells have differentiated, more preferably less than 10% of the cell population have differentiated, and most preferably less than 5% of the cell population have differentiated.

The term “thawing” as used herein refers to the process of increasing the temperature of a cryopreserved cell, embryo, or portions of animals. Methods of thawing cryopreserved materials such that they are active after the thawing process are well-known to those of ordinary skill in the art.

The term “dissociating” as used herein refers to the materials and methods useful for pulling a cell away from another cell. For example, a blastomere (i.e., a cellular member of a morula or blastocyst stage embryo) can be pulled away from the rest of the developing cell mass by techniques and apparatus well known to a person of ordinary skill in the art. See, e.g., U.S. Pat. No. 4,994,384, entitled “Multiplying Bovine Embryos,” issued on Feb. 19, 1991, hereby incorporated herein by reference in its entirety, including all figures, tables, and drawings. Alternatively, cells proliferating in culture can be separated from one another to facilitate such processes as cell passaging, which is described previously. In addition, dissociation of a cultured cell from a group of cultured cells can be useful as a first step in the process of nuclear transfer, as described hereafter. When a cell is dissociated from an embryo, the dissociation manipulation can be useful for such processes as re-cloning, a process described herein, as well as a step for multiplying the number of embryos.

The term “non-embryonic cell” as used herein refers to a cell that is not isolated from an embryo. Non-embryonic cells can be differentiated or non-differentiated. Non-embryonic cells refers to nearly any somatic cell or any germ line cell, such as cells isolated from an ex utero animal. These examples are not meant to be limiting.

The term “fetus” as used herein refers to a developing cell mass that has implanted into the uterine membrane of a maternal host. A fetus can include such defining features as a genital ridge, for example. A genital ridge is a feature easily identified by a person of ordinary skill in the art, and is a recognizable feature in fetuses of most animal species. The term “fetal cell” as used herein refers to any cell isolated from and/or has arisen from a fetus or derived from a fetus. The term “non-fetal cell” is a cell that is not derived or isolated from a fetus.

When cells are isolated from a fetus, such cells are preferably isolated from fetuses where the fetus is between 20 days and parturition, between 30 days and 100 days, more preferably between 35 days and 70 days and between 40 days and 60 days, and most preferably about a 55 day fetus. An age of a fetus can be determined from the time that an embryo, which develops into the fetus, is established. The term “about” with respect to fetuses refers to plus or minus five days.

The term “parturition” as used herein refers to a time that a fetus is delivered from female recipient. A fetus can be delivered from a female recipient by abortion, c-section, or birth.

In preferred embodiments, the cells and cell lines of the instant invention are primary cells, cultured cells, embryonic cells, non-embryonic cells, fetal cells, genital ridge cells, primordial germ cells, embryonic germ cells, embryonic stem cells, somatic cells, adult cells, fibroblasts, differentiated cells, undifferentiated cells, amniotic cells, ovarian follicular cells, and cumulus cells. Preferably, such cells grow to confluent monolayers in culture.

The term “primordial germ cell” as used herein refers to a diploid somatic cell capable of becoming a germ cell. Primordial germ cells can be isolated from the genital ridge of a developing cell mass. The genital ridge is a section of a developing cell mass that is well-known to a person of ordinary skill in the art. See, e.g., Strelchenko, 1996, Theriogenology 45: 130-141 and Lavoir 1994, J. Reprod. Dev. 37: 413-424. Such cells, when cultured, are referred to by the skilled artisan as “embryonic germ cells.”

The term “embryonic stem cell” as used herein refers to pluripotent cells isolated from an embryo that are maintained in in vitro cell culture. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells are well known to a person of ordinary skill in the art. See, e.g., WO 97/37009, entitled “Cultured Inner Cell Mass Cell-Lines Derived from Ungulate Embryos,” Stice & Golueke, published Oct. 9, 1997, and Yang & Anderson, 1992, Theriogenology 38: 315-335, both of which are incorporated herein by reference in their entireties, including all figures, tables, and drawings.

The term “differentiated cell” as used herein refers to a cell that has developed from an unspecialized phenotype to that of a specialized phenotype. For example, embryonic cells can differentiate into an epithelial cell lining the intestine. It is highly unlikely that differentiated cells revert into their precursor cells in vivo or in vitro. However, materials and methods of the invention can reprogram differentiated cells into immortalized, totipotent cells. Differentiated cells can be isolated from a fetus or a live born animal, for example.

The term “undifferentiated cell” as used herein refers to a cell that has an unspecialized phenotype and is capable of differentiating. An example of an undifferentiated cell is a stem cell.

The term “asynchronous population” as used herein refers to cells that are not arrested at any one stage of the cell cycle. Many cells can progress through the cell cycle and do not arrest at any one stage, while some cells can become arrested at one stage of the cell cycle for a period of time. Some known stages of the cell cycle are G₀, G₁, S, G₂, and M. An asynchronous population of cells is not manipulated to synchronize into any one or predominantly into any one of these phases. Cells can be arrested in the G₀ stage of the cell cycle, for example, by utilizing multiple techniques known in the art, such as by serum deprivation. Examples of methods for arresting non-immortalized cells in one part of the cell cycle are discussed in WO 97/07669, entitled “Quiescent Cell Populations for Nuclear Transfer,” hereby incorporated herein by reference in its entirety, including all figures, tables, and drawings.

The terms “synchronous population” and “synchronizing” as used herein refer to a fraction of cells in a population that are arrested (i.e., the cells are not dividing) in a discrete stage of the cell cycle. Preferably, about 50% of the cells in a population of cells are arrested in one stage of the cell cycle, more preferably about 70% of the cells in a population of cells are arrested in one stage of the cell cycle, and most preferably about 90% of the cells in a population of cells are arrested in one stage of the cell cycle. Cell cycle stage can be distinguished by relative cell size as well as by a variety of cell markers well known to a person of ordinary skill in the art. For example, cells can be distinguished by such markers by using flow cytometry techniques well known to a person of ordinary skill in the art. Alternatively, cells can be distinguished by size utilizing techniques well known to a person of ordinary skill in the art, such as by the utilization of a light microscope and a micrometer, for example.

The term “adult cell” as used herein refers to a cell from a live-born animal.

The term “amniotic cell” as used herein refers to any cultured or non-cultured cell isolated from amniotic fluid. Examples of methods for isolating and culturing amniotic cells are discussed in Bellow et al., 1996, Theriogenology 45: 225; Garcia & Salaheddine, 1997, Theriogenology 47:1003-1008; Leibo & Rail, 1990, Theriogenology 33: 531-552; and Vos et al., 1990, Vet. Rec. 127: 502-504, each of which is incorporated herein by reference in its entirety, including all figures tables and drawings. Particularly preferred are cultured amniotic cells that are rounded (e.g., cultured amniotic cells that do not display a fibroblast-like morphology). Also preferred amniotic cells are fetal fibroblast cells. The terms “fibroblast,” fibroblast-like,” “fetal,” and “fetal fibroblast” are defined hereafter.

The term “fibroblast” as used herein refers to cultured cells having a flattened and elongated morphology that are able to grow in monolayers. Preferably, fibroblasts grow to confluent monolayers in culture. While fibroblasts characteristically have a flattened appearance when cultured on culture media plates, fetal fibroblast cells can also have a spindle-like morphology. Fetal fibroblasts may require density limitation for growth, may generate type I collagen, and may have a finite life span in culture of approximately fifty generations. Preferably, fetal fibroblast cells rigidly maintain a diploid chromosomal content. For a description of fibroblast cells, see, e.g., Culture of Animal Cells: a manual of basic techniques (3^{rd} edition), 1994, R. I. Freshney (ed), Wiley-Liss, Inc., incorporated herein by reference in its entirety, including all figures, tables, and drawings.

The term “uterine cell” as used herein refers to any cell isolated from a uterus. Preferably, a uterine cell is a cell deriving from a pregnant adult animal. In preferred embodiments, uterine cells are cells obtained from fluid that fills the uterine cavity. Such cells can be obtained by numerous methods well known in the art such as amniocentesis.

The term “ovarian follicular cell” as used herein refers to a cultured or non-cultured cell obtained from an ovarian follicle, other than an oocyte. Follicular cells may be isolated from ovarian follicles at any stage of development, including primordial follicles, primary follicles, secondary follicles, growing follicles, vesicular follicles, maturing follicles, mature follicles, and graafian follicles. Furthermore, follicular cells may be isolated when an oocyte in an ovarian follicle is immature (i.e., an oocyte that has not progressed to metaphase II) or when an oocyte in an ovarian follicle is mature (i.e., an oocyte that has progressed to metaphase II or a later stage of development). Preferred follicular cells include, but are not limited to, pregranulosa cells, granulosa cells, theca cells, columnar cells, stroma cells, theca interna cells, theca externa cells, mural granulosa cells, luteal cells, and corona radiata cells. Particularly preferred follicular cells are cumulus cells. Various types of follicular cells are known and can be readily distinguished by those skilled in the art. See, e.g., Laboratory Production of Cattle Embryos, 1994, Ian Gordon, CAB International; Anatomy and Physiology of Farm Animals (5th ed.), 1992, R. D. Frandson and T. L. Spurgeon, Lea & Febiger, each of which is incorporated herein by reference in its entirety including all figures, drawings, and tables. Individual types of follicular cells may be cultured separately, or a mixture of types may be cultured together.

The term “cumulus cell” as used herein refers to any cultured or non-cultured cell isolated from cells and/or tissue surrounding an oocyte. Persons skilled in the art can readily identify cumulus cells. Examples of methods for isolating and/or culturing cumulus cells are discussed in Damiani et al., 1996, Mol. Reprod. Dev. 45: 521-534; Long et al., 1994, J. Reprod. Fert. 102: 361-369; and Wakayama et al., 1998, Nature 394: 369-373, each of which is incorporated herein by reference in its entireties, including all figures, tables, and drawings. Cumulus cells may be isolated from ovarian follicles at any stage of development, including primordial follicles, primary follicles, secondary follicles, growing follicles, vesicular follicles, maturing follicles, mature follicles, and graafian follicles. Cumulus cells may be isolated from oocytes in a number of manners well known to a person of ordinary skill in the art. For example, cumulus cells can be separated from oocytes by pipeting the cumulus cell/oocyte complex through a small bore pipette, by exposure to hyaluronidase, or by mechanically disrupting (e.g. vortexing) the cumulus cell/oocyte complex. Additionally, exposure to Ca⁺⁺/Mg⁺⁺ free media can remove cumulus from immature oocytes. Also, cumulus cell cultures can be established by placing matured oocytes in cell culture media.

The term “nuclear donor” as used herein refers to any cell, or nucleus thereof, having nuclear DNA that can be translocated into an oocyte. A nuclear donor may be a nucleus that has been isolated from a cell. Multiple techniques are available to a person of ordinary skill in the art for isolating a nucleus from a cell and then utilizing the nucleus as a nuclear donor. See, e.g., U.S. Pat. Nos. 4,664,097, 6,011,197, and 6,107,543, each of which is hereby incorporated by reference in its entirety including all figures, tables and drawings. Any type of cell can serve as a nuclear donor. Examples of nuclear donor cells include, but are not limited to, cultured and non-cultured cells isolated from an embryo arising from the union of two gametes in vitro or in vivo; embryonic stem cells (ES cells) arising from cultured embryonic cells (e.g., pre-blastocyst cells and inner cell mass cells); cultured and non-cultured cells arising from inner cell mass cells isolated from embryos; cultured and non-cultured pre-blastocyst cells; cultured and non-cultured fetal cells; cultured and non-cultured adult cells; cultured and non-cultured primordial germ cells; cultured and non-cultured germ cells (e.g., embryonic germ cells); cultured and non-cultured somatic cells isolated from an animal; cultured and non-cultured cumulus cells; cultured and non-cultured amyniotic cells; cultured and non-cultured fetal fibroblast cells; cultured and non-cultured genital ridge cells; cultured and non-cultured differentiated cells; cultured and non-cultured cells in a synchronous population; cultured and non-cultured cells in an asynchronous population; cultured and non-cultured serum-starved cells; cultured and non-cultured permanent cells; and cultured and non-cultured totipotent cells. See, e.g., Piedrahita et al., 1998, Biol. Reprod. 58: 1321-1329; Shim et al., 1997, Biol. Reprod. 57: 1089-1095; Tsung et al., 1995, Shih Yen Sheng Wu Hsueh Pao 28: 173-189; and Wheeler, 1994, Reprod. Fertil. Dev. 6: 563-568, each of which is incorporated herein by reference in its entirety including all figures, drawings, and tables. In addition, a nuclear donor may be a cell that was previously frozen or cryopreserved.

The term “activation” refers to any materials and methods useful for stimulating a cell to divide before, during, and after a nuclear transfer step. Cybrids may require stimulation in order to divide after a nuclear transfer has occurred. The invention pertains to any activation materials and methods known to a person of ordinary skill in the art. Although electrical pulses are sometimes sufficient for stimulating activation of cybrids, other means are sometimes useful or necessary for proper activation of the cybrid. Chemical materials and methods useful for activating embryos are described below in other preferred embodiments of the invention.

Examples of non-electrical means for activation include agents such as ethanol; inositol trisphosphate (IP₃); Ca⁺⁺ ionophores (e.g., ionomycin) and protein kinase inhibitors (e.g., 6-dimethylaminopurine (DMAP)); temperature change; protein synthesis inhibitors (e.g., cyclohexamide); phorbol esters such as phorbol 12-myristate 13-acetate (PMA); mechanical techniques; and thapsigargin. The invention includes any activation techniques known in the art. See, e.g., U.S. Pat. No. 5,496,720, entitled “Parthenogenic Oocyte Activation” to Susko-Parrish et al., issued on Mar. 5, 1996; and U.S. Pat. No. 6,077,710, issued on Jun. 20, 2000, each of which is incorporated by reference herein in its entirety, including all figures, tables, and drawings.

The term “fusion” as used herein in reference to nuclear transfer refers to the combination of portions of lipid membranes corresponding to the nuclear donor and the recipient oocyte. Lipid membranes can correspond to the plasma membranes of cells or nuclear membranes, for example. The fusion can occur between the nuclear donor and recipient oocyte when they are placed adjacent to one another, or when the nuclear donor is placed in the perivitelline space of the recipient oocyte, for example. Specific examples for translocation of the totipotent mammalian cell into the oocyte are described hereafter in other preferred embodiments. These techniques for translocation are fully described in the references cited previously herein in reference to nuclear transfer.

The term “electrical pulses” as used herein with respect to fusion of cells in nuclear transfer refers to subjecting the nuclear donor and recipient oocyte to electric current. For nuclear transfer, the nuclear donor and recipient oocyte can be aligned between electrodes and subjected to electrical current. The electrical current can be alternating current or direct current. The electrical current can be delivered to cells for a variety of different times as one pulse or as multiple pulses. The cells are typically cultured in a suitable medium for the delivery of electrical pulses. Examples of electrical pulse conditions utilized for nuclear transfer are described in the references and patents previously cited herein in reference to nuclear transfer.

The term “fusion agent” as used herein in reference to nuclear transfer refers to any compound or biological organism that can increase the probability that portions of plasma membranes from different cells will fuse when a totipotent mammalian cell nuclear donor is placed adjacent to the recipient oocyte. In preferred embodiments fusion agents are selected from the group consisting of polyethylene glycol (PEG), trypsin, dimethylsulfoxide (DMSO), lectins, agglutinin, viruses, and Sendai virus. These examples are not meant to be limiting and other fusion agents known in the art are applicable and included herein.

The term “suitable concentration” as used herein in reference to fusion agents, refers to any concentration of a fusion agent that affords a measurable amount of fusion. Fusion can be measured by multiple techniques well known to a person of ordinary skill in the art, such as by utilizing a light microscope, dyes, and fluorescent lipids, for example.

The term “totipotent” as used herein refers to a cell, embryo, or fetus capable of giving rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps. Totipotent cells, embryos, and fetuses may also be used to generate incomplete animals such as those useful for organ harvesting, e.g., having genetic modifications to eliminate growth of an organ or appendage by manipulation of a homeotic gene.

The term “live born” as used herein preferably refers to an animal that exists ex utero. A “live born” animal may be an animal that is alive for at least one second from the time it exits the maternal host. A “live born” animal may not require the circulatory system of an in utero environment for survival. A “live born” animal may be an ambulatory animal. Such animals can include pre- and post-pubertal animals. As discussed previously, a live born animal may lack a portion of what exists in a normal animal of its kind.

The term “isolated” as used herein in reference to cells refers to a cell that is mechanically separated from another group of cells. Examples of a group of cells are a developing cell mass, a cell culture, a cell line, and an animal. These examples are not meant to be limiting and the invention relates to any group of cells. Methods for isolating one or more cells from another group of cells are well known in the art. See, e.g., Culture of Animal Cells: a manual of basic techniques (3^{rd} edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. Morgan, John Wiley and Sons, Ltd.

For the purposes of the present invention, the terms “embryo” or “embryonic” as used herein refer to a developing cell mass that has not implanted into the uterine membrane of a maternal host. Hence, the term “embryo” as used herein can refer to a fertilized oocyte, a cybrid (defined herein), a pre-blastocyst stage developing cell mass, and/or any other developing cell mass that is at a stage of development prior to implantation into the uterine membrane of a maternal host. Embryos of the invention may not display a genital ridge. Hence, an “embryonic cell” is isolated from and/or has arisen from an embryo.

An embryo can represent multiple stages of cell development. For example, a one cell embryo can be referred to as a zygote, a solid spherical mass of cells resulting from a cleaved embryo can be referred to as a morula, and an embryo having a blastocoel can be referred to as a blastocyst.

The terms “enucleated oocyte” or “enucleated recipient cell” as used herein refer to an oocyte which has had its nucleus removed. Typically, a needle can be placed into an oocyte and the nucleus can be aspirated into the inner space of the needle. The needle can be removed from the oocyte without rupturing the plasma membrane. This enucleation technique is well known to a person of ordinary skill in the art. See, U.S. Pat. No. 4,994,384; U.S. Pat. No. 5,057,420; and Willadsen, 1986, Nature 320:63-65. An enucleated oocyte can be prepared from a young or an aged oocyte. An enucleated oocyte is preferably prepared from an oocyte that has been matured, in vitro or in vivo, for some period of time. This time can vary, depending on the source species for the oocyte. For example, bovine oocytes are preferably matured for between 10 hours and 40 hours, more preferably for between 16 hours and 36 hours, and most preferably between 20 hours and 32 hours. Enucleation at 17-21 hrs, transfer about an hour later. Range 10-48, 16-36. In contrast, porcine oocytes are preferably matured for greater than 24 hours, and more preferably matured for greater than 36 hours. In particularly preferred embodiments, a porcine oocyte is matured for more than 40 hours, up to about 96 hours, more preferably from 42-54 hours, and even more preferably from 42 to 48 hours.

The terms “maturation” and “matured” as used herein refer to the process in which an oocyte is incubated. Oocytes can be incubated in vitro with multiple media well known to a person of ordinary skill in the art. See, e.g., Saito et al., 1992, Roux's Arch. Dev. Biol. 201: 134-141 for bovine organisms and Wells et al., 1997, Biol. Repr. 57: 385-393 for ovine organisms and also Mattioli et al., 1989, Theriogenology 31: 1201-1207; Jolliff & Prather, 1997, Biol. Reprod. 56: 544-548; Funahashi & Day, 1993, J. Reprod. Fert. 98: 179-185; Nagashima et al., 1997, Mol. Reprod. Dev. 38: 339-343; Abeydeera et al., 1998, Biol. Reprod. 58: 213-218; Funahashi et al., 1997, Biol. Reprod. 57: 49-53; and Sawai et al., 1997, Biol. Reprod. 57: 1-6, each of which are incorporated herein by reference in their entireties including all figures, tables, and drawings. Maturation media can comprise multiple types of components, including microtubule inhibitors (e.g., cytochalasin B), hormones and growth factors. Other examples of components that can be incorporated into maturation media are discussed in WO 97/07668, entitled “Unactivated Oocytes as Cytoplast Recipients for Nuclear Transfer,” Campbell & Wilmut, published on Mar. 6, 1997, hereby incorporated herein by reference in its entirety, including all figures, tables, and drawings. The time of maturation can be determined from the time that an oocyte is placed in a maturation medium to the time that the oocyte is subject to a manipulation (e.g., enucleation, nuclear transfer, fusion, and/or activation).

Oocytes can be matured for any period of time: an oocyte can be matured for greater than 10 hours, greater than 20 hours, greater than 24 hours, greater than 36 hours, greater than 48 hours, greater than 60 hours, greater than 72 hours, and greater than 90 hours. The term “about” with respect to oocyte maturation refers to plus or minus 3 hours.

An oocyte can also be matured in vivo. Time of maturation may be the time that an oocyte receives an appropriate stimulus to resume meiosis to the time that the oocyte is manipulated by enucleation. Similar maturation periods described.above for in vitro matured oocytes apply to in vivo matured oocytes.

Nuclear transfer may be accomplished by combining one nuclear donor and more than one enucleated oocyte. In addition, nuclear transfer may be accomplished by combining one nuclear donor, one or more enucleated oocytes, and the cytoplasm of one or more enucleated oocytes.

The term “young oocyte” as used herein refers to an oocyte that has been matured in vitro for a time less than or equal to the length of time between the onset of estrus and ovulation in vivo. For example, the onset of estrus is signaled by a surge in leutenizing hormone. A cow typically ovulates about 26 hours following the onset of estrus. Thus, a young oocyte is an oocyte matured for about 26 hours or less, preferably 16 to 17 hours. Methods for measuring the length of time between the onset of estrus and ovulation are well known to the skilled artisan. See, e.g., P. T. Cupps, “Reproduction in Domestic Animals,” Fourth Edition, Academic Press, San Diego, Calif., USA, 1991. For horses, ovulation occurs about 33 hours after onset of estrus; for pigs, about 40 hours; for sheep and goats, about 24-36 hours; for dogs, about 40-50 hours; and for cats, about 24-36 hours. The term ” young oocyte” may also refer to an oocyte that has been matured and ovulated in vivo and that is collected at about the time of ovulation. The term about in this context refers to +/−1 hour.

Oocytes can be isolated from live animals using methods well known to a person of ordinary skill in the art. See, e.g., Pieterse et al., 1988, “Aspiration of bovine oocytes during transvaginal ultrasound scanning of the ovaries,” Theriogenology 30: 751-762. Oocytes can be isolated from ovaries or oviducts of deceased or live born animals. Suitable media for in vitro culture of oocytes are well known to a person of ordinary skill in the art. See, e.g., U.S. Pat. No. 5,057,420, which is incorporated by reference herein.

Some young oocytes can be identified by the appearance of their ooplasm. Because certain cellular material (e.g., lipids) have not yet dispersed within the ooplasm. Young oocytes can have a pycnotic appearance. A pycnotic appearance can be characterized as clumping of cytoplasmic material. For example, in bovines, a “pycnotic” appearance is to be contrasted with the appearance of oocytes that are older than 28 hours, which have a more homogenous appearing ooplasm.

The term “aged oocyte” as used herein refers to an oocyte that has been matured in vitro for a time greater than the length of time between the onset of estrus and ovulation in vivo. The term “aged oocyte” may also refer to an oocyte that has been matured and ovulated in vivo and that is collected later than about 1 hour after the time of ovulation. An aged oocyte can be identified by its characteristically homogenous ooplasm. This appearance is to be contrasted with the pycnotic appearance of young oocytes as described previously herein. The age of the oocyte can be defined by the time that has elapsed between the time that the oocyte is placed in a suitable maturation medium and the time that the oocyte is activated. The age of the oocyte can dramatically enhance the efficiency of nuclear transfer. For example, an aged oocyte can be more susceptible to activation stimuli than a young oocyte.

The term “ovulated in vivo” as used herein refers to an oocyte that is isolated from an animal a certain number of hours after the animal exhibits characteristics that it is in estrus. The characteristics of an animal in estrus are well known to a person of ordinary skill in the art, as described in references disclosed herein.

The terms “maternal recipient” and “recipient female” as used herein refer to a female animal which is implanted with an embryo for development of the embryo. A maternal recipient may be either homospecific or xenospecific to the implanted embryo. For example it has been shown in the art that bovine embryos can develop in the oviducts of sheep. Stice & Keefer, 1993, “Multiple generational bovine embryo cloning,” Biology of Reproduction 48: 715-719. Implanting techniques are well known to a person of ordinary skill in the art. See, e.g., Polge & Day, 1982, “Embryo transplantation and preservation,” Control of Pig Reproduction, D J A Cole and G R Foxcroft, eds., London, UK, Butterworths, pp. 227-291; Gordon, 1997, “Embryo transfer and associated techniques in pigs,” Controlled reproduction in pigs (Gordon, ed), CAB International, Wallingford UK, pp 164-182; and Kojima, 1998, “Embryo transfer,” Manual of pig embryo transfer procedures, National Livestock Breeding Center, Japanese Society for Development of Swine Technology, pp 76-79, each of which is incorporated herein by reference in its entirety, including all figures, tables, and drawings.

The term “transgenic” as used herein in reference to embryos, fetuses and animals refers to an embryo, fetus or animal comprising one or more cells that contain heterologous nucleic acids. In preferred embodiments, a transgenic embryo, fetus, or animal comprises one or more transgenic cells. While germ line transmission is not a requirement of transgenic embryos, fetuses, or animals as that term is used herein, in particularly preferred embodiments a transgenic embryo, fetus, or animal can pass its transgenic characteristic(s) through the germ line. In certain embodiments, a transgenic embryo, fetus or animal expresses one or more transgenes as transgenic RNA and protein molecules. Most preferably, a transgenic embryo, fetus or animal results from a nuclear transfer procedure using a transgenic nuclear donor cell.

The terms “milk protein promoter,” “urine protein promoter,” “blood protein promoter,” “tear duct protein promoter,” “synovial protein promoter,” “spermatogenesis protein promoter,” and “mandibular gland protein promoter” refer to promoter elements that regulate the specific expression of proteins within the specified fluid or gland or cell type in an animal. For example, a milk protein promoter is a regulatory element that can control the expression of a protein that is expressed in the milk of an animal. Other promoters, such as β-casein promoter, melanocortin promoter, milk serum protein promoter, casein promoter, α-lactalbumin promoter, whey acid protein promoter, uroplakin promoter, and α-actin promoter, for example, are well known to a person of ordinary skill in the art.

The terms “insertion” and “introduction” as used herein in reference to artificial chromosomes or other large heterologous nucleic acid constructs refer to translocating one or more such artificial chromosomes or constructs from the outside of a cell to the inside of a cell. Insertion can be effected in at least two manners: by mechanical delivery and non-mechanical delivery.

The term “mechanical delivery” as used herein refers to processes that utilize an apparatus that directly or indirectly introduces DNA (e.g., one or more artificial chromosomes) into one or more cells. Examples of mechanical delivery of DNA into cells include, but are not limited to, microinjection, particle bombardment, sonoporation, and electroporation.

The summary of the invention described above is not limiting and other features and advantages of the invention will be apparent from the following detailed description of the preferred embodiments, as well as from the claims.

### DETAILED DESCRIPTION OF THE INVENTION

The present invention provides techniques and compositions that allow for the substantial replication or reproduction of an immune response present in a first non-human founder animal in a clone of the founder animal. The immune response of interest according to the present invention is a response to one or more antigens of interest. Typically, the immune response will comprise the production of antibodies against the antigen or antigens of interest. In this embodiment, the present invention allows a supply of a valuable antibody to be maintained after the initial antibody-producing animal is no longer capable of providing the antibodies against the antigen or antigens of interest. Because the unique attributes of the antibodies produced by the founder animal in response to the antigen or antigens of interest may depend on the polyclonality of the antibodies (i.e. the animal's serum may contain antibodies of different IgG sub-isotypes with specificities for different epitopes on the antigen(s) of interest that together contribute to the serum's unique performance characteristics), the founder animal's antibody production characteristics and resulting antibody profile are preferably maintained with as little variation and adulteration as possible. After the clone of the founder animal is prompted to produce an antibody profile that is substantially identical to that of the founder animal, the antibodies can be isolated and purified for sale or use as reagents in imnmunoassays.

Alternatively, the immune response can embody any immune response, such as for example swelling, itching, allergy, anaphylaxis, arthritis, autoimmune disorders or the like, so that useful animal models of disease states and conditions can be propagated and maintained when one animal is recognized as having an immune response of interest. Providing a plurality of animals which are models for disease or conditions are useful for diagnosing, treating and testing for the genetic basis of the disease or condition.

In certain preferred embodiments, the first step in replicating an immune response of a first, preferably non-human, animal comprises utilizing the animal as a founder mammal for the creation of one or more clones of the founder animal. Preferably a plurality of clones of the founder animal are created so that a potentially large number of animals which are virtually genetically identical to the founder mammal are available in which to replicate the immune response of interest of the founder animal. After the founder animal has been successfully cloned, the clone or clones can be conditioned so that an immune response to the antigen(s) of interest in the clone that is substantially identical to the founder animal's immune response to the same antigen or antigens is produced. Any animal which can be successfully cloned is suitable for use in the present invention. Particularly preferred animals for use in the present invention are ungulates, including sheep, cows, pigs and goats.

In order to achieve the desired immune response in the clone, several strategies are available. First, the clones can be subjected to the same environmental conditions and stresses as the founder animal. Preferably, the desired immune response of the clone to the antigen(s) of interest can also be achieved by subjecting the cloned offspring to the same immunization strategy used on the founder animal. More preferably, the cloned animals are raised under the same environmental factors as the founder animal and subjected to the same immunization strategy as that of the founder animal. Immunization strategies for various animals are described, e.g., in Hanley et al., “Review of Polyclonal Antibody Production Procedures in Mammals and Poultry,” ILAR Journal 37: 93-118 (1995).

These strategies depend primarily on the genetic contribution towards the specific desired immune response in a mammal, preferably to a vaccination regimen. It is well established that there is a significant genetic contribution to specific immune responses. The heritable nature of susceptibility to specific infectious or autoimmune diseases is also well established. The most definitive evidence for the importance of genetic factors in a primary immune response comes from the study of twins. Because of genetic identity, studies of monozygotic (identical) twins are an appropriate model for consideration of cloned animals. Identical twins, similar to clones, may differ genetically, for example, in terms of immune response differences in somatic rearrangement during development of the T-cell antigen receptor and antibody repertoire. Such differences may result from a number of epigenetic factors during development, and result in differing immune responses. However, specific antibody responses in monozygotic twins compared to dizygotic (fraternal) twins illustrate an important genetic influence, at least on amount of antibody produced. See, Konradsen et al., Clin. Exp. Immunol. 92(3):532-536 (1993). Several studies in sheep have been published, analyzing the effect of genetic factors on resistance to parasitic infection, or on antibody response to specific immunogens. See, e.g., Stear and Murray, Vet. Parasitol. 54:161-176 (1994); Shu et al., Vet. Res. Commun. January 2001;25(1):43-54 (2001). Overall these studies provide convincing evidence that in cloned animals the specific immune response to an antigen is likely to be similar at least of isotype and total amount of antibody produced.

After the substantially identical immune response to the antigen of interest in the clone has been achieved, the clone preferably produces antibodies against the one or more antigens of interest. Once the cloned animal produces the antibodies against the antigen(s) of interest these antibodies can be isolated and purified from the clone for further use.

Alternatively, one or more of the cells of the immune system of the founder animal can be adoptively transferred into the clone to maintain the immune response in these recipient clones. Preferably, the immune system cells adoptively transferred to the cloned animal comprise one or more of the following cell types: B-lymphocytes, T-lymphocytes, antibody secreting cells (ASC) and memory cells. More preferably, the production of antibodies by the adoptively transferred immune system cells is maintained by immunization of the cloned animal with the same or similar vaccinations as were given to the founder animal.

It is well known in the art that allografts do not survive because they are rejected by the recipient's immune system, a response primarily mediated by T cells. This makes it impossible to transplant a component of an animal's immune system to another animal without first ablating the recipient's immune system using radiation or an agent such as 5-fluorouracil. Even successful grafts typically cannot be properly maintained without the chronic administration of immunosuppressive agents. Moreover, in genetically distinct animals, the immune graft may attack the cells of the recipient animal in a process known as “graft versus host” disease.

Such problems can be avoided entirely according to the present invention because with the availability of cloned offspring of the founder animal, the immune system cell transplants are effectively autologous thus virtually eliminating the possibility of graft versus host disease (GVHD) or host versus graft disease (HVGD) rejection. Accordingly, the direct transplantation of immune system cells, such as lymphocytes, into recipient cloned animals is possible. Although typically not necessary, in order to allow for expansion and maintenance of the transplanted immune system cells it may be necessary to partially or fully myeloablate the lymphocyte population of the recipient clone, and to drive proliferation of one or more populations of transferred immune cells by vaccination within a few days after transplantation. Immune system cell ablation is well known in the art and can occur using a variety of means, including radiation and/or chemical agents such as 5-fluorouracil. An advantage enjoyed by the present inventive method is that the T cell population of the recipient may recognize the antigen presenting MHC II molecules of the donor (founder animal) memory B cells, allowing for T-cell-dependent help and cytokine production.

The present invention allows for the immune system cells of the founder animal to be donated to the acceptor clone at any point during the life cycle of the acceptor clone. Preferably, the immune system cells of the founder animal are transplanted into the acceptor clone after the immune system of the acceptor clone has undergone some post-natal maturation and maternal immunity has partially waned (8-12 weeks in rabbit, 3-6 weeks of age in sheep). Administering the donor immune system cells from the founder animal at an early age may avoid the administration of lymphocyte ablation treatments. Additionally, a vaccination regime may be begun simultaneously with the transplantation of the immune system cells or shortly thereafter.

Preferred immune system cells for adoptive transfer to the recipient clone include the cells responsible for the desirable immune response of the founder animal, non-limiting examples of which include long-lived ASC resident in lymphoid compartments. These ASC cells have differentiated germ line DNA with VDJ rearrangements that encode the antigen specificity of the immunoglobulin molecules they produce. Terminally differentiated ASC are called plasma cells, and these cells have a very high rate of antibody production (estimated to be greater than 5000 antibody molecules per second). Plasma cells differ from memory cells in that plasma cells are non-dividing and have lost all surface-bound immunoglobulin. Memory cells express surface immunoglobulin molecules, and respond to antigenic stimulation by proliferating and differentiating into plasma cells and additional memory B cells. Through the expression of surface immunoglobulin molecules, MEC II and co-stimulatory molecules (such as B-7), memory B cells are very efficient antigen presenting cells and help to maintain ASC numbers. Recognized sites of T-cell-dependent antibody responses include the germinal centers of the spleen and the lymph nodes. It has been shown that long-term immunity to viral infection depends on chronic antibody production by plasma cells primarily resident in the bone marrow. However, it remains likely that memory cells, with the capacity to proliferate and regenerate both themselves and the plasma cell population, are primarily resident in the lymphoid organ draining the original site of vaccination i.e. lymph nodes.

Preferred sources for harvesting of immune cells for adoptive transfer include lymph nodes, spleen, liver, and bone marrow. Peripheral blood mononuclear cells (PBMCs) are an alternative source of immune system cells for transplantation. PBMCs offer a considerable advantage in that they can be simply collected in quite large numbers with minimal intervention. However, appropriate precursor ASC will likely be present at a very low frequency in PBMCs, making them a less preferred source of cells for transplantation. Due to the fact that memory cells are primarily present in the lymph nodes, together with their easy accessibility for surgical removal, the lymph nodes draining vaccination sites are preferred targets for harvesting of cells for adoptive transfer according to the present invention.

Once immune system cells for transplantation are harvested from the founder mammal, the immune system cells can be expanded to provide a larger number cells for transplantation thereby increasing the change of successful engraftment. Techniques for proliferation of immune system cells are well known in the art. For example, proliferation of memory cells and ASC's can be accomplished by in vitro antigen pulsing and addition of recombinant IL-2 possibly combined with treatment with anti-CD3. Such treatment can result in considerable expansion of cell numbers and increase the prospects of successful transfer of ASC.

In a preferred embodiment, several lymph nodes (i.e. prescapular, popliteal) are surgically removed from the founder mammal under general anaesthetic within 7-10 days of an immunization. The removed lymph nodes should then be completely dissociated under sterile conditions and resuspended at 1×10⁷ cells/ml in RPMI medium (supplemented with L-glutamine, antibiotics) plus 10% fetal bovine serum, optionally including 10% DMSO if the cells are to undergo cryopreservation. The harvested immune system cells can be either directly transplanted into the recipient clone or frozen for later use in adoptive transfer procedures. Excellent viability can be anticipated on subsequent thawing of these cells and very high cell numbers can be prepared from a single lymph node. Preferably, serum from the founder animal should also be administered to the recipient clone at the time of the immune system cell transplantation, as there is evidence that inclusion of antibody containing serum supports effective engraftment. Serum may also be collected from the founder animal and cryopreserved for later use.

Administration of the immune system. cells harvested from the founder animal to the clone can be performed by numerous methods as is well known in the art. Although intravenous administration of the harvested immune system cells is preferred, cells may be directly transferred to various sites in the acceptor animal (e.g., bone marrow, spleen, thymus, and the lymphatic system). Effective amounts of immune system cells to be administered to the recipient clone can be readily determined by one of ordinary skill in the art with routine experimentation. Preferably, at least 1×10¹⁰ immune system cells are adoptively transferred to the recipient clone to achieve the present invention.

I. Nuclear Transfer Cloning

Nuclear transfer (NT) techniques are well known to a person of ordinary skill in the art. See, e.g., U.S. Pat. No. 4,664,097, “Nuclear Transplantation in the Mammalian Embryo by Microsurgery and Cell Fusion,” issued May 12, 1987, McGrath & Solter; U.S. Pat. No. 4,994,384 (Prather et al.); 5,057,420 (Massey et al.); U.S. Pat. No. 6,107,543; U.S. Pat. No. 6,011,197; Proc. Nat'l. Acad. Sci. USA 96: 14984-14989 (1999); Nature Genetics 22: 127-128 (1999); Cell & Dev. Diol 10: 253-258 (1999); Nature Biotechnology 17: 456-461 (1999); Science 289: 1188-1190 (2000); Nature Biotechnol. 18: 1055-1059 (2000); and Nature 407: 86-90 (2000); each of which is incorporated herein by reference in its entirety.

A. Nuclear Donors

For NT techniques, a donor cell may be separated from a growing cell mass, isolated from a primary cell culture, or isolated from a cell line. The entire cell may be placed in the perivitelline space of a recipient oocyte or may be directly injected into the recipient oocyte by aspirating the nuclear donor into a needle, placing the needle into the recipient oocyte, releasing the nuclear donor and removing the needle without significantly disrupting the plasma membrane of the oocyte. Also, a nucleus (e.g., karyoplast) may be isolated from a nuclear donor and placed into the perivitelline space of a recipient oocyte or may be injected directly into a recipient oocyte, for example.

Any cell can be used in principle as a nuclear donor cell. Examples of nuclear donor cells include, but are not limited to, cultured and non-cultured cells isolated from an embryo arising from the union of two gametes in vitro or in vivo; embryonic stem cells (ES cells) arising from cultured embryonic cells (e.g., pre-blastocyst cells and inner cell mass cells); cultured and non-cultured cells arising from inner cell mass cells isolated from of embryos; cultured and non-cultured pre-blastocyst cells; cultured and non-cultured fetal cells; cultured and non-cultured primordial germ cells; cultured and non-cultured embryonic germ cells; cultured and non-cultured B-cells, cultured and non-cultured T-cells; cultured and non-cultured somatic cells isolated from an animal; cultured and non-cultured non-somatic cells isolated from an animal; cultured and non-cultured cumulus cells; cultured and non-cultured amniotic cells; cultured and non-cultured fetal fibroblast cells; cultured and non-cultured genital ridge cells; cultured and non-cultured differentiated cells; cultured and non-cultured cells in a synchronous population; cultured and non-cultured cells in an asynchronous population; cultured and non-cultured serum-starved cells; cultured and non-cultured permanent cells; and cultured and non-cultured totipotent cells. See, e.g., Piedrahita et al., 1998, Biol. Reprod. 58: 1321-1329; Shim et al., 1997, Biol. Reprod. 57: 1089-1095; Tsung et al., 1995, Shih Yen Sheng Wu Hsueh Pao 28: 173-189; and Wheeler, 1994, Reprod. Fertil. Dev. 6: 563-568, each of which is incorporated herein by reference in its entirety including all figures, drawings, and tables. In addition, a nuclear donor may be a cell that was previously frozen or cryopreserved.

In particularly preferred embodiments, a nuclear donor cell is a transgenic cell. The term “transgenic” as used herein in reference to cells refers to a cell whose genome has been altered using recombinant DNA techniques. In preferred embodiments, a transgenic cell comprises one or more exogenous DNA sequences in its genome. In other preferred embodiments, a transgenic cell comprises a genome in which one or more endogenous genes have been deleted, duplicated, activated, or modified. In particularly preferred embodiments, a transgenic cell comprises a genome having both one or more exogenous DNA sequences, and one or more endogenous genes that have been deleted, duplicated, activated, or modified.

When an immune cell, such as a B-cell or T-cell is used as a nuclear donor, the adoptive transfer and/or immunization procedures described herein may or may not be necessary. This is because such cells contain rearranged immunoglobulin or T-cell receptor gene sequences within their genome. By carefully selecting the donor cell to represent the immune response of interest (e.g., a B-cell containing rearranged immunoglobulin sequences that produce an antibody of interest), each cell in the resulting clone will contain that rearrangement. In effect, a monoclonal antibody producer may be created. It is also possible that, because such cells contain a second immunoglobulin gene allele, that an additional antibody repertoire may also be created within the animal.

The present invention also relates to methods and compositions that can advantageously increase the efficiencies of nuclear transfer procedures. In particular, cells, prefereably cultured cells, to be used as a source of nuclear donor material are contacted with one or more compounds that affect cholesterol biosynthesis prior to the use of the cell, or its nucleus, as a nuclear donor. Preferably, such a compound is an inhibitor of an enzyme in the cholesterol biosynthesis pathway.

HMG-CoA reductase is a key early step in the synthesis of cholesterol, and numerous compounds have been identified that inhibit this enzyme. Thus, HMG-CoA reductase represents an attractive target for the methods of the instant invention. Numerous inhibitors of HMG-CoA reductase, such as lovastatin, simvistatin, pravastatin, fluvastatin, atorvastatin, and cerivastatin, and methods of producing such compounds, are known in the art. These compounds have found utility in reducing serum cholesterol in humans. For example, lovastatin is hydrolyzed in vivo to a β-hydroxyacid metabolite, which is an active and specific inhibitor of HMG-CoA reductase. Similarly, pravastatin is administered as an active sodium ester, which is a competitive inhibitor of HMG-CoA reductase:

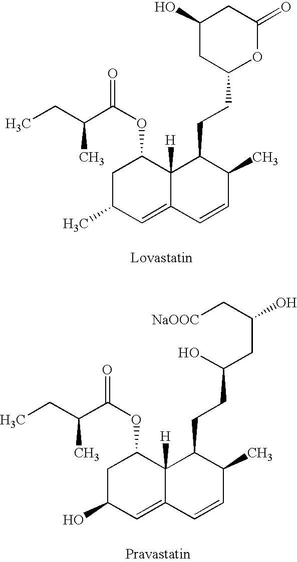

A cell to be used as a source of nuclear donor material can be contacted with one or more inhibitors of an enzyme in the cholesterol biosynthesis pathway (e.g., HMG-CoA reductase), such as statins. Such contacting can be performed, for example, by incubating the cell in a medium comprising one or more inhibitors of HMG-CoA reductase. The cell can be contacted with such an inhibitor for any length of time and at any concentration of inhibitor. Preferably, the cell is contacted with a concentration of the inhibitor, and for a length of time, effective for the inhibitor to enter the cell and inhibit HMG-CoA reductase; however, the actual time of contacting and concentration used is most preferably selected based on its ability to increase the efficiency of a nuclear transfer procedure, as determined by the percentage of nuclear transfer embryos that reach cleavage stage, that reach fetal stage, and/or that result in a live-born animal.

Preferred concentrations of statins are from about 0.05 μM to about 500 μM; more preferred concentrations are from about 0.5 μM to about 50 μM; and most preferred concentrations are about 5 μM. The actual concentration required to provide a beneficial effect can depend on the ability of a given statin to enter the cell, or whether the statin must be metabolized to provide an active inhibitor, etc.

Once the cell has been incubated in the inhibitor-containing medium for an appropriate period of time, the cell (or its nucleus or nuclear contents) can be transferred to a recipient cell in a nuclear transfer procedure as described herein.

B. Recipient Cells

A recipient cell is typically an oocyte with a portion of its ooplasm removed, where the removed ooplasm comprises the oocyte nucleus or nuclear DNA. Enucleation techniques are well known to a person of ordinary skill in the art. See e.g., Nagashima et al., 1997, Mol. Reprod. Dev. 48: 339-343; Nagashima et al., 1992, J. Reprod. Dev. 38: 37-78; Prather et al., 1989, Biol. Reprod. 41: 414-418; Prather et al., 1990, J. Exp. Zool. 255: 355-358; Saito et al., 1992, Assis. Reprod. Tech. Andro. 259: 257-266; and Terlouw et al., 1992, Theriogenology 37: 309, each of which is incorporated herein by reference in its entirety including all figures, tables, and drawings. Cells other than oocytes can also be successfully used as recipient cells. See, e.g., Polejaeva et al., Nature 407(6800): 86-90 (2000).

Oocytes can be isolated from either oviducts and/or ovaries of live animals by oviductal recovery procedures or transvaginal oocyte recovery procedures well known in the art and described herein. Furthermore, oocytes can be isolated from deceased animals. For example, ovaries can be obtained from abattoirs and oocytes can be aspirated from these ovaries. The oocytes can also be isolated from the ovaries of a recently sacrificed animal or when the ovary has been frozen and/or thawed.

Oocytes can be matured in a variety of media well known to a person of ordinary skill in the art. One example of such a medium suitable for maturing oocytes is depicted in an exemplary embodiment described hereafter. Oocytes can be successfully matured in this type of medium within an environment comprising 5% CO₂ at 37-39° C., preferably 38° C. Oocytes may be cryopreserved and then thawed before placing the oocytes in maturation medium. Cryopreservation procedures for cells and embryos are well known in the art as discussed herein.

Components of an oocyte maturation medium can include molecules that arrest oocyte maturation. Examples of such components are 6-dimethylaminopurine (DMAP) and isobutylmethylxanthine (IBMX). IBMX has been reported to reversibly arrest oocytes, but the efficiencies of arrest maintenance are quite low. See, e.g., Rose-Hellkant and Bavister, 1996, Mol. Reprod. Develop. 44: 241-249. However, oocytes may be arrested at the germinal vesicle stage with a relatively high efficiency by incubating oocytes at 31° C. in an effective concentration of IBMX. Preferably, oocytes are incubated the entire time that oocytes are collected. Concentrations of IBMX suitable for arresting oocyte maturation are 0.01 mM to 20 mM IBMX, preferably 0.05 mM to 10 mM IBMX, and more preferably about 0.1 mM IBMX to about 5 mM IBMX, and most preferably 0.1 mM IBMX to 0.5 mM IBMX. In certain embodiments, oocytes can be matured in a culture environment having a low oxygen concentration, such as 5% O₂, 5-10% CO₂, and 85-90% N₂.

A nuclear donor cell and a recipient oocyte can arise from the same species or different species. For example, a totipotent porcine cell can be inserted into a porcine enucleated oocyte. Alternatively, a totipotent wild boar cell can be inserted into a domesticated porcine oocyte. Any nuclear donor/recipient oocyte combinations are envisioned by the invention. Preferably the nuclear donor and recipient oocyte are from the same specie. In certain embodiments, the nuclear donor and recipient oocyte are both derived from the founder animal provided of course that the founder animal is female. Cross-species NT techniques can be utilized to produce cloned animals that are endangered or extinct.

Oocytes can be activated by electrical and/or non-electrical means before, during, and/or after a nuclear donor is introduced to recipient oocyte. For example, an oocyte can be placed in a medium containing one or more components suitable for non-electrical activation prior to fusion with a nuclear donor. Also, a cybrid can be placed in a medium containing one or more components suitable for non-electrical activation. Activation processes are discussed in greater detail hereafter.

C. Injection/Fusion

A nuclear donor cell or nucleus can be translocated into an oocyte using a variety of materials and methods that are well known to a person of ordinary skill in the art. In one example, a nuclear donor cell or nucleus may be directly injected into a recipient oocyte. This direct injection can be accomplished by gently pulling a nuclear donor cell or nucleus into a needle, piercing a recipient oocyte with that needle, releasing the nuclear donor material into the oocyte, and removing the needle from the oocyte without significantly disrupting its membrane. Appropriate needles can be fashioned from glass capillary tubes, as defined in the art and specifically by publications incorporated herein by reference.

In another example, at least a portion of plasma membrane from a nuclear donor and recipient oocyte can be fused together by utilizing techniques well known to a person of ordinary skill in the art. See, Willadsen, 1986, Nature 320:63-65, hereby incorporated herein by reference in its entirety including all figures, tables, and drawings. Typically, lipid membranes can be fused together by electrical and chemical means, as defined previously and in other publications incorporated herein by reference.

Examples of non-electrical means of cell fusion involve incubating cybrids in solutions comprising polyethylene glycol (PEG), and/or Sendai virus. PEG molecules of a wide range of molecular weight can be utilized for cell fusion.

Processes for fusion that are not explicitly discussed herein can be determined without undue experimentation. For example, modifications to cell fusion techniques can be monitored for their efficiency by viewing the degree of cell fusion under a microscope.

D. Activation

Methods of activating oocytes and cybrids are known to those of ordinary skill in the art. See, U.S. Pat. 5,496,720, “Parthenogenic Oocyte Activation,” Susko-Parrish et al., issued on Mar. 5, 1996, hereby incorporated by reference herein in its entirety including all figures, tables, and drawings.

Both electrical and non-electrical processes can be used for activating cells (e.g., oocytes and cybrids). Although use of a non-electrical means for activation is not always necessary, non-electrical activation can enhance the developmental potential of cybrids, particularly when young oocytes are utilized as recipient cells.

Examples of electrical techniques for activating cells are well known in the art. See, WO 98/16630, published on Apr. 23, 1998, Piedraheidra and Blazer, hereby incorporated herein in its entirety, and U.S. Pat. Nos. 4,994,384 and 5,057,420. Non-electrical means for activating cells can include any method known in the art that increases the probability of cell division. Examples of non-electrical means for activating a nuclear donor and/or recipient can be accomplished by introducing cells to ethanol; inositol trisphosphate (IP₃); Ca²⁺ ionophore such as ionomycin or A23187; protein kinase inhibitors such as 6-dimethylaminopurine; temperature change; protein synthesis inhibitors (e.g., cycloheximide); phorbol esters such as phorbol 12-myristate 13-acetate (PMA); mechanical techniques; thapsigargin; sperm factors; or a combination of the above. Sperm factors can include any component of a sperm that enhance the probability for cell division. Other non-electrical methods for activation include subjecting the cell or cells to cold shock and/or mechanical stress.

Examples of preferred protein kinase inhibitors are protein kinase A, G, and C inhibitors such as 6-dimethylaminopurine (DMAP), staurosporin, 2-aminopurine, sphingosine. Tyrosine kinase inhibitors may also be utilized to activate cells.

Activation materials and methods that are not explicitly discussed herein can be identified by modifying the specified conditions defined in the exemplary protocols described hereafter and in U.S. Pat. No. 5,496,720.

E. Manipulation of Embryos Resulting from Nuclear Transfer

An embryo resulting from a NT process can be manipulated in a variety of manners. The invention relates to cloned embryos that arise from at least one NT. Exemplary embodiments of the invention demonstrate that two or more serial NT procedures may enhance the efficiency for the production of totipotent embryos.

When multiple serial NT procedures are utilized for the formation of a cloned totipotent embryo, oocytes that have been matured for any period of time can be utilized as recipients in the first, second or subsequent NT procedures. Additionally, one or more of the NT cycles may be preceded, followed, and/or carried out simultaneously with an activation step. As defined previously herein, an activation step may be accomplished by electrical and/or non-electrical means as defined herein. Exemplified embodiments described hereafter describe NT techniques that incorporate an activation step after one NT cycle. However, an activation step may also be carried out at the same time as a NT cycle (e.g., simultaneously with the NT cycle) and/or an activation step may be carried out prior to a NT cycle. Cloned totipotent embryos resulting from a NT cycle can be (1) disaggregated or (2) allowed to develop further.

If embryos are disaggregated, disaggregated embryonic derived cells can be utilized to establish cultured cells. Any type of embryonic cell can be utilized to establish cultured cells. These cultured cells are sometimes referred to as embryonic stem cells or embryonic stem-like cells in the scientific literature. The embryonic stem cells can be derived from early embryos, morulae, and blastocyst stage embryos. Multiple methods are known to a person of ordinary skill in the art for producing cultured embryonic cells. These methods are enumerated in specific references previously incorporated by reference herein.

If embryos are allowed to develop into a fetus in utero, cells isolated from that developing fetus can be utilized to establish cultured cells. In preferred embodiments, primordial germ cells, genital ridge cells, and fetal fibroblast cells can be isolated from such a fetus. Cultured cells having a particular morphology that is described herein can be referred to as embryonic germ cells (EG cells). These cultured cells can be established by utilizing culture methods well known to a person of ordinary skill in the art. Such methods are enumerated in publications previously incorporated herein by reference and are discussed herein. In particularly preferred embodiments, Streptomyces griseus protease can be used to remove unwanted cells from the embryonic germ cell culture.

Cloned totipotent embryos resulting from NT can also be manipulated by cryopreserving and/or thawing the embryos. See, e.g., Nagashima et al., 1989, Japanese J. Anim. Reprod. 35: 130-134 and Feng et al., 1991, Theriogenology 35: 199, each of which is incorporated herein by reference in its entirety including all tables, figures, and drawings. Other embryo manipulation methods include in vitro culture processes; performing embryo transfer into a maternal recipient; disaggregating blastomeres for NT processes; disaggregating blastomeres or inner cell mass cells for establishing cell lines for use in NT procedures; embryo splitting procedures; embryo aggregating procedures; embryo sexing procedures; and embryo biopsying procedures. The exemplary manipulation procedures are not meant to be limiting and the invention relates to any embryo manipulation procedure known to a person of ordinary skill in the art.

II. Development of Cloned Embryos

A. Culture of Embryos In Vitro

Cloning procedures discussed herein provide an advantage of culturing cells and embryos in vitro prior to implantation into a recipient female. Methods for culturing embryos in vitro are well known to those skilled in the art. See, e.g., Nagashima et al., 1997, Mol. Reprod. Dev. 48: 339-343; Petters & Wells, 1993, J. Reprod. Fert. (Suppl) 48: 61-73; Reed et al., 1992, Theriogenology 37: 95-109; and Dobrinsky et al., 1996, Biol. Reprod. 55: 1069-1074, each of which is incorporated herein by reference in its entirety, including all figures, tables, and drawings. In addition, exemplary embodiments for media suitable for culturing cloned embryos in vitro are described hereafter. Feeder cell layers may or may not be utilized for culturing cloned embryos in vitro. Feeder cells are described hereafter and in exemplary embodiments hereafter.

B. Development of Embryos In Utero

Cloned embryos can be cultured in an artificial or natural uterine environment after NT procedures and embryo in vitro culture processes. Examples of artificial development environments are being developed and some are known to those skilled in the art. Components of the artificial environment can be modified, for example, by altering the amount of a component or components and by monitoring the growth rate of an embryo.

Methods for implanting embryos into the uterus of an animal are also well known in the art, as discussed hereafter. Preferably, the developmental stage of the embryo(s) is correlated with the estrus cycle of the animal.

Embryos from one species can be placed into the uterine environment of an animal from another species. For example it has been shown in the art that bovine embryos can develop in the oviducts of sheep. Stice & Keefer, 1993, “Multiple generational bovine embryo cloning,” Biology of Reproduction 48: 715-719. The invention relates to any combination of a mammalian embryo in any other mammalian uterine environment. A cross-species in utero development regime can allow for efficient production of cloned animals of an endangered species. For example, a wild boar embryo can develop in the uterus of a domestic porcine sow.

Once an embryo is placed into the uterus of a recipient female, the embryo can develop to term. Alternatively, an embryo can be allowed to develop in the uterus and then can be removed at a chosen time. Surgical methods are well known in the art for removing fetuses from uteri before they are born.

III. Chimeric Animals

The materials and methods described herein can also be used to derive chimeric animals that reproduce an immune response. Methods for making chimeric animals are well known to those of skill in the art. See, e.g., U.S. Pat. No. 5,994,619. In these methods, one or more cells, e.g., from an embryo, are introduced into a second embryo, resulting in a chimeric embryo that may be implanted into a maternal host.

For example, a B-cell or T-cell may be used as a nuclear donor to produce a nuclear transfer embryo. As discussed above, such donor cells contain rearranged immunoglobulin or T-cell receptor gene sequences within their genome. Cells from this embryo may be directly introduced into a second embryo, which has been produced by either fertilization or nuclear transfer methods. By selecting the B- or T-cell to represent the immune response of interest (e.g., a B-cell containing rearranged immunoglobulin sequences that produce an antibody of interest), the resulting clone will contain cells expressing that rearrangement, in a background of “normal” cells.

Moreover, the chimeric embryo can be created that express multiple cell types in a “normal” cell background. For example, an 8-cell embryo may contain 4 different introduced cell types and 4 cells from the recipient embryo. In this way, much of the immune response of interest from a founder animal can be introduced into a recipient embryo, while the recipient embryo provides an unmanipulated immune system able to provide a response to a broader immune challenge.

### EXAMPLES

The examples below are not limiting and are merely representative of various aspects and features of the present invention.

### Example 1

### Transfer and Replication of an Immune Response

A. Harvesting of Immune Cells from the Founder Animal (Sheep)

An animal is placed under general anaesthesia 7 to 10 days following an immunization. Several lymph nodes that drain the sites of immunization (e.g., prescapular, popliteal) are surgically removed.

Lymph nodes are dissociated mechanically or enzymatically under sterile conditions and lymphocytes are resuspended at 1×10⁷ cells per milliliter in RPMI medium (containing L-glutamine and antibiotics) supplemented with 10% FBS and 10% DMSO as a cryopreservative. The cells are then frozen and stored at −70° C. to −196° C.

Antibody-containing serum is collected from the founder animal by venapuncture for later intravenous administration to the clone and also frozen. Antibody-containing serum may support effective engraftment (Merica et al., 164(9):4551-7 (2000)).

The target cell for transfer is the memory B-cell. Memory B-cells express immunoglobulin molecules on their surface and are stimulated by immunogen to proliferate and produce terminally differentiated antibody producing cells, i.e., plasma cells, and additional memory B-cells. Memory B-cells are likely to be found in lymph nodes draining the sites of immunization, in peripheral blood and in bone marrow.

B. Adoptive Transfer of Lymph Node Cells to the Clone(s) of the Founder Animal

Three- to six-week old clones of the founder animal are administered cells (1×10¹⁰) from the dissociated lymph nodes. Cells are administered by intravenous injection in saline and/or founder serum. Immunization of clones at this age allows for post-natal maturation of the clone's immune system coincident with diminution of maternal immunity.

C. Adoptive Transfer of Peripheral Blood Cells to the Clone(s) of the Founder Animal

Peripheral blood mononuclear cells (PBMCs) are an alternative source for transplantation. A low frequency of memory B-cells in this population, however, may require that peripheral blood be “pulsed” with immunogen in order to expand these populations prior to adoptive transfer. Peripheral blood is collected from the founder animal by venapuncture. Blood cells are cultured with immunogen and recombinant ovine IL-2 and anti-CD3. Three- to six-week old clones of the founder animal are administered cells (1×10¹⁰) from the pulsed blood cell cultures. Cells are administered by intravenous injection in saline and/or in founder serum. Immunization of clones at this age allows for post-natal maturation of the clone's immune system coincident with diminution of maternal immunity.

Immunization of the Clones following Adoptive Transfer of Lymphocytes

The immunization protocol for the clone begins coincidentally with the adoptive transfer of lymphocytes and follows the original immunization protocol of the founder animal. Thus, the specificity of the immunogen, its nativity, injection solution(s), quantity, and the route and frequency of its injection will vary according to the existing protocol for each founder animal.

### Example 2

### Cloning Porcine Animals

Cells suitable for establishing a cloned porcine animal can be can be established from nearly any cell type. For example, fibroblast or fibroblast-like cell cultures are established from ear punches extracted from a selected animal; and cultured fibroblast or fibroblast-like cells are established from fetuses. Individual cells isolated from such a cell culture are then utilized as nuclear donors in a nuclear transfer process. A single nuclear transfer cycle or multiple nuclear transfer cycles can be applied.

Day 41 to day 60 porcine fetuses were collected from pregnant gilts. The intact uterus was excised from the gilt and immediately transported to the laboratory for recovery of fetuses. Fetal gender, weight, crown-rump length and individual identification were recorded prior to dissection. Genital ridge cells were obtained by 0.3% protease (from S. griseus) digestion of the genital ridges for 45 minutes at 37° C. Body cells were obtained from a partial body trypsin-EDTA (Life Technologies, Grand Island, N.Y.) digest (minus head and viscera) for 45 minutes at 37° C. Following digestion, cells were filtered through a 70 μm cell strainer (BD Biosciences), counted and suspended in high glucose Dulbecco Modified Eagle Medium (DMEM, Life Technologies) supplemented with 10% fetal bovine serum (FBS, Hyclone, Logan, Utah) and 0.1 mM β-mercaptoethanol and transferred to 35 mm tissue culture dishes (Nalge Nunc, Naperville, Ill.) at 1×10⁵-10×10⁵ cells/ml. Typically, donor cells were passaged into 4-well plates (Nalge Nunc) and grown to confluence. Immediately prior to nuclear transfer, donor cells in one well were dissociated by incubation with 0.1% protease for approximately 10 minutes, washed once with TL-HEPES supplemented with 10% FBS, collected by centrifugation for 10 minutes at 250×g and resuspended in approximately 0.5 ml Dulbecco PBS (DPBS, Life Technologies).

Porcine Oocyte Recovery and Maturation

Sow and gilt ovaries were collected at separate, local abattoirs and maintained at 30° C. during transport to the laboratory. Follicles ranging from 2-8 mm were aspirated into 50 ml conical centrifuge tubes (BD Biosciences, Franklin Lakes, N.J.) using 18 gauge needles and vacuum set at 100 mm of mercury. Follicular fluid and aspirated oocytes from sows and gilts were pooled separately and rinsed through EmCono® filters (Iowa Veterinary Supply Company, Iowa Falls, Iowa) with HEPES buffered Tyrodes solution (Biowhittaker, Walkersville, Md.). Oocytes surrounded by a compact cumulus mass were selected and placed into North Carolina State University (NCSU) 37 oocyte maturation medium (Petters et al., J Reprod Fertil Suppl 48, 61-73 (1993)) supplemented with 0.1 mg/ml cysteine (Grupen et al., Biol Reprod 53, 173-178 (1995)), 10 ng/ml EGF (epidermal growth factor) (Grupen et al., Reprod Fertil Dev 9, 571-575 (1997)), 10% PFF (porcine follicular fluid) (Naito et al., Gamete Res 21, 289-295 (1988)), 0.5 mg/ml cAMP (Funahashi et al., Biol Reprod 57, 49-53 (1997)), 10 IU/ml each of PMSG (pregnant mare serum gonadotropin) and hCG (human chorionic gonadotropin) for approximately 22 hours (Funahashi et al., J Reprod Fertil 98, 179-185 (1993)) in humidified air at 38.5° C. and 5% CO₂. Subsequently, they were moved to fresh NCSU 37 maturation medium which did not contain cAMP, PMSG or hCG and incubated for an additional 22 hours. After approximately 44 hours in maturation medium, oocytes were stripped of their cumulus cells by vortexing in 0.1% hyaluronidase for 1 minute. Sow and gilt derived oocytes were each used in the in vitro fertilization and nuclear transfer procedures described below. These procedures were controlled so that comparisons could be made between sow and gilt derived oocytes for in vitro embryo development, pregnancy initiation rate upon embryo transfer, and litter size upon farrowing.

Nuclear Transfer

Upon removal of cumulus cells, oocytes were placed in CR2 (Rosenkranz et al., Theriogenology 35, 266 (1991)) embryo culture medium that contained 1 μg/ml Hoechst 33342 and 7.5 μg/ml cytochalasin B for approximately 30 minutes. Micromanipulation of oocytes was performed using glass capillary microtools in 150 μl drops of TL HEPES on 100 mm dishes (BD Biosciences) covered with light mineral oil. Glass capillary microtools were produced using a pipette puller (Sutter Instruments, Novato, Calif.) and microforge (Narishige International, East Meadow N.Y.). Metaphase II oocytes were enucleated by removal of the polar body and the associated metaphase plate. Absence of the metaphase plate was visually verified by ultraviolet fluorescence, keeping exposure to a minimum A single donor cell obtained from a confluent culture was placed in the perivitelline space of the oocyte so as to contact the oocyte membrane. A single electrical pulse of 95 volts for 45 μsec from an ElectroCell Manipulator 200 (Genetronics, San Diego, Calif.) was used to fuse the membranes of the donor cell and oocyte, forming a cybrid. The fusion chamber consisted of wire electrodes 500 um apart and the fusion medium was SOR2 (0.25 M sorbitol, 0.1 mM calcium acetate, 0.5 mM magnesium acetate, 0.1% BSA, pH 7.2, and osmolarity 250). Following the fusion pulse, cybrids were incubated in CR2 embryo culture medium for approximately 4 hours prior to activation.

Activation

Oocytes/cybrids were activated by incubation in 15 μM calcium ionomycin (Calbiochem, San Diego, Calif.) for 20 minutes followed by incubation with 1.9 mM 6-dimethylaminopurine (DMAP) in CR2 for 3-4 hours. After DMAP incubation, cybrids were washed through two 35 mmn plates containing TL-HEPES, cultured in CR2 medium containing BSA (3 mg/ml) for 48 hours, then placed in NCSU 23 medium containing 0.4% BSA for 24 hours followed by a final culture in NCSU 23 containing 10% FBS. Selected embryos that developed to blastocyst stage by day 7 in vitro were fixed (4% paraformaldehyde), stained with Hoechst 33342 and placed under cover slips on glass slides. Fixed embryos were visualized with ultraviolet fluorescence and cells were counted.

Embryo Transfer and Pregnancy Detection

Embryos at various stages of development were surgically transferred into uteri of asynchronous recipients essentially as described by Rath (Rath et al., Theriogenology 47, 795-800 (1997)). Briefly, recipients (parity 0 or 1 female porcines) were selected that exhibited first standing estrus from 24 hours prior to oocyte activation to 24 hours following oocyte activation. For surgical embryo transfer, recipients were anesthetized with a combination of 2 mg/kg ketamine, 0.25 mg/kg tiletamine/zolazepam, 1 mg/kg xylazine and 0.03 mg/kg atropine (Iowa Veterinary Supply). Anesthesia was maintained with 3% halothane (Iowa Veterinary Supply). While in dorsal recumbence, the recipients were aseptically prepared for surgery and a caudal ventral incision was made to expose and examine the reproductive tract. Embryos that were cultured less than 48 hours (1-2 cell stage) were generally placed in the ampullar region of the oviduct by feeding a 5.5-inch TomCat® catheter (Sherwood Medical) through the ovarian fimbria. Embryos cultured 48 hours or more (≧4 cell stage) were generally placed in the tip of the uterine horn using a similar catheter. Typically, 100-400 NT embryos were placed in the oviduct or uterine tip, depending on embryonic stage and 100 IVF embryos were placed in the oviduct. All recipients and protocols conformed to University of Wisconsin animal health-care guidelines. Ultrasound detection of pregnancy was accomplished using an Aloka 500 ultrasound scanner (Aloka Co. Ltd, Wallingford, Conn.) with an attached 3.5 MHz trans-abdominal probe. Monitoring for pregnancy initiation began at 23 days post fusion/fertilization and repeated as necessary through day 40. Pregnant recipients were reexamined by ultrasound weekly.

### Example 3

### Cloning Bovine Animals

Feeder Layer Preparation

A feeder cell layer was prepared from mouse fetuses that were from 10 to 20 days gestation. The head, liver, heart and alimentary tract were removed and the remaining tissue washed and incubated at 37° C. in 0.05% trypsin-0.53 mM EDTA (Gibco, Cat #25300-54). Loose cells were cultured in tissue culture dishes containing MEM-alpha medium (Gibco Cat #32561-037) supplemented with penicillin (100 units/ml), streptomycin (100 μg/ml), 10% fetal bovine serum and 0.1 mM 2-mercaptoethanol. The feeder cell cultures were cultured for one to three weeks at 37° C., 5% CO₂ and humidified air. Before being used as feeder cells, the cells were pre-treated with mitomycin C (Calbiochem, Cat #47589) at a final concentration of 10 μg/ml for 3 hours and washed 5 times with PBS before pre-equilibrated growth media was added.

Feeder cells can be established from bovine, porcine, or ovine fetuses from 30 to 70 days using the same procedure. Such fetal cells may be optionally treated with mitomycin C.

Establishing Cultured Cells from Non-Embryonic Tissue

As discussed above for porcines, virtually any type of bovine precursor cell can be used to generate totipotent bovine cells for use in nuclear transfer. Such precursor cells can be embryonic cells, cultured embryonic cells, primordial germ cells, fetal cells, and cells isolated from the tissues of adult animals, for example. For example, cumulus cells isolated from the ovary and ear cells from an adult bovine have been utilized as precursor cells for the generation of totipotent cells.

A first step towards generating totipotent cells from tissues of grown animals includes a primary culture of isolated cells. A protocol for culturing cells isolated from the tissues of grown animals is provided hereafter. Although the illustrative protocol relates to ear punch samples, this protocol can apply to cells isolated from any type of tissue.

The following steps are preferably performed utilizing sterile procedures:

1) Wash each ear sample twice with 2 mL of trypsin/EDTA solution (0.05% trypsin-0.53 mM EDTA (Gibco, Cat #25300-54) in two separate 35 mm Petri dishes. Process each ear sample separately. Mince the ear sample with sterile scissors and scalpel in a 35 mm Petri dish containing 2 mL of trypsin/EDTA solution. The minced pieces are preferably less than 1 mm across.

2) Incubate minced ear pieces in the trypsin/EDTA solution for 40-50 min. in a 37 C incubator with occasional swirling. The dish may be wrapped with a stretchable material, such as Parafilm®, to reduce CO2 accumulation.

3) Transfer digested ear pieces to a 15 mL sterile tube. Wash the dish from which the digested ear pieces were recovered with 2 mL of the trypsin/EDTA solution and transfer this wash solution to the sterile tube.

4) Vortex the tube at medium speed for 2 min.

5) Add 5 mL of media (defined below) to inactivate the trypsin.

6) Centrifuge the 15 mL tube at 280×g for 10 minutes.

7) Aspirate the supernatant and re-suspend the cell pellet in residual solution by gently taping the side of the tube.

8) Add 2 mL of media to the tube and then centrifuge as described in step (6).

9) Aspirate the supernatant, re-suspend the pellet as described in step (7), then add 2 mL of media.

10) Keep 2-3 pieces of the ear for DNA analysis and store at −20 C.

11) Transfer resuspended cells into a 35 mm Nunc culture dish and incubate in medium at 37° C. in a humidified 5% CO₂/95% air atmosphere.

12) Change media every2 days.

Medium:

Combine Alpha minimum essential medium (MEM) (Life Technologies Cat #32561-037) with 10% fetal bovine serum (Hyclone), 100 U/mL penicillin, 100 μg/mL streptomycin, 0.25 μg/mL amphotercin B (Fungizone).

This protocol has been also successfully utilized to establish cultures of kidney and liver cells isolated from grown bovine animals. As discussed above, the protocol can be utilized to create cell cultures from any type of cell isolated from a grown animal, for any species or family of animals.

As another example, the following procedure describes one embodiment of the invention, where primordial germ cells were utilized as precursor cells for the generation of totipotent cells.

Bovine fetuses approximately 40-80 days old were obtained from pregnant animals. The genital ridges were located at the caudo-ventral part of the abdominal cavity. Genital ridges were removed aseptically and washed in phosphate buffered saline (PBS) (Gibco, Cat #14287-015) with 500 U/mL penicillin/500 μg/ml streptomycin. The tissue was sliced into 1-1.5 mm pieces and placed into a solution containing pronase E (3 mg/ml; Sigma Cat #P6911) in Tyrodes Lactate (TL) HEPES (Biowhittaker, Cat #04-616F) for 30-45 minutes at 35-37° C. The proteolytic action of pronase E disaggregated the slices of genital ridges to a cell suspension. Pronase E was removed by dilution and centrifugation in TL HEPES solution. After this step, the cell suspension was cultured as described below, or frozen and stored at −196° C.

A fresh or thawed cell suspension (final concentration 1×10⁵-10×10⁵ cells/ml) was placed into a 35 mm Petri dish containing a murine primary embryonic fibroblast feeder layer. The culture media used was MEM alpha (Life Technologies Cat #32561-037) supplemented with 0.1 mM 2-mercaptoethanol (Gibco, Cat #21985-023), 25-100 ng/ml human recombinant leukemia inhibitory factor (hrLIF; R&D System, Cat #250-L), 100 ng/ml bovine basic fibroblast growth factor (bFGF; R&D System, Cat #133-FB) and 10% fetal calf serum (FCS, HyClone) at 37.5° C. and 5% CO₂. Alternatively, AmnioMax medium plus supplement (Life Technologies Cat #'s 27000-025 &) was used without a feeder layer. Exogenous steel factor (e.g., membrane associated steel factor and soluble steel factor) was not added to the culture media.

After 24 hours, and again at 48 hour intervals, supplemented culture media was replaced. After an initial culture of 6 days in MEM alpha, concentrations of hrLIF and bFGF were lowered if appropriate to 25-40 ng/ml. After nine days in culture, hrLIF and bFGF were removed from the medium entirely.

Embryo Construction

The following embodiment of the invention describes materials and methods utilized to produce totipotent embryos of the invention. Embryos of the invention can be produced by utilizing totipotent cells of the invention as nuclear donors in NT procedures. As described previously, multiple NT procedures can be utilized to create a totipotent embryo. The following two examples describe a multiple NT procedure, which describes the use of two NTs.

Mycoplasma free totipotent cells used in the NT procedure, were prepared by cutting out a group of cells from the culture dish using a glass needle. The cells were then incubated in a TL HEPES solution containing from 1 to 3 mg/ml pronase E at approximately 32° C. for 15-60 minutes, the amount of time which was needed in this example to disaggregate the cells. Once the cells were in a single cell suspension they were used for NT within a 2-3 hour period.

Oocytes aspirated from ovaries were matured overnight (16 hours) in maturation medium. Medium 199 (Biowhittaker, Cat #12-119F) supplemented with luteinizing hormone 10 IU/ml (LH; Sigma, Cat #L9773), 1 mg/ml estradiol (Sigma, Cat #E8875) and 10% FCS or estrus cow serum, was used. Within 16-17 hours of maturation, the cumulus layer expanded and the first polar bodies were extruded.

In the first NT procedure, young oocytes (16-17 hours in maturation medium) were stripped of their cumulus cell layers and nuclear material stained with Hoechst 33342 5 mg/ml (Sigma, Cat #2261) in TL HEPES solution supplemented with cytochalasin B (7 μg/ml, Sigma, Cat #C6762) for 15 min. Oocytes were then enucleated in TL HEPES solution under mineral oil. A single cell of optimal size (12 to 15 μm) was then selected from a cell suspension and injected into the perivitelline space of the enucleated oocyte. The cell and oocyte membranes were then induced to fuse by electrofusion in a 500 μm chamber by application of an electrical pulse of 90V for 15 μs.

Cybrid activation was induced by a 4 min exposure to 5 μM calcium ionophore A23187 (Sigma Cat. #C-7522) or ionomycin Ca-salt in HECM (hamster embryo culture medium) containing 1 mg/ml BSA followed by a 1:1000 dilution in HECM containing 30 mg/ml BSA for 5 min. For HECM medium, See, e.g., Seshagiri & Barister, 1989, “Phosphate is required for inhibition of glucose of development of hamster eight-cell embryos in vitro,” Biol. Reprod. 40: 599-606. This step is followed by incubation in CR2 medium containing 1.9 mM 6-dimethylaminopurine (DMAP; Sigma product, Cat #D2629) for 4 hrs followed by a wash in HECM and then cultured in CR2 media with BSA (3 mg/ml) under humidified air with 5% CO₂ at 39° C. For CR2 medium, See, e.g., Rosenkrans & First, 1994, “Effect of free amino acids and vitamins on cleavage and developmental rate of bovine zygotes in vitro,” J. Anim. Sci. 72: 434-437. Mitotic divisions of the cybrid formed an embryo. Three days later the embryos were transferred to CR2 media containing 10% FCS for the remainder of their in vitro culture.

Second Nuclear Transfer (Recloning)

Embryos from the first generation NT at the morula stage were disaggregated either by pronase E (1-3 mg/ml in TL HEPES) or mechanically after treatment with cytochalasin B. Single blastomeres were placed into the perivitelline space of enucleated aged oocytes (28-48 hours in maturation medium). Aged oocytes were produced by incubating matured “young” oocytes for an additional time in CR2 media with 3 mg/ml BSA in humidified air with 5% CO₂ at 39° C.

A blastomere from an embryo produced from the first NT procedure was fused into the enucleated oocyte via electrofusion in a 500 μm chamber with an electrical pulse of 105V for 15 μs in an isotonic sorbitol solution (0.25 M sorbitol, 0.1 mM calcium acetate, 0.5 mM magnesium acetate, 0.1% BSA, pH 7.2; osmolarity=250) at 30° C. Aged oocytes were simultaneously activated with a fusion pulse, not by chemical activation as with young oocytes.

After blastomere-oocyte fusion, the cybrids from second generation NT were cultured in CR2 media supplemented with BSA (3 mg/ml) under humidified air with 5% CO₂ at 39° C. On the third day of culture, developing embryos were evaluated and cultured further until day seven in CR2 media containing 10% FCS. Morphologically good to fair quality embryos were non-surgically transferred into recipient females.

### Example 4

### Cloning Ovine Animals

Oocyte Collection and Maturation

Oocytes were aspirated from sheep ovaries obtained from an abattoir and recovered in TL HEPES medium (Biowhittaker 04-616F) containing 10 mg/ml Heparin (Sigma H-3393) and 4 mg/ml BSA (Sigma A-6003). Aspirations were performed using 20 GA needles with the vacuum set at 60 mm Hg.

Two maturation media were used to produce nuclear transfer pregnancies. Maturation Medium 1: TC199 (Gibco 11150-059), 2 mM Glutamine (Sigma G-5763), 10% FBS (Hyclone A-111D), 5 mg/ml ovine FSH (Sigma L-8174), 5 mg/ml ovine LH (Sigma L-5269), 1 mg/ml Estradiol (Sigma E-2257), 0.3 mM Na-pyruvate (Sigma P-4562), and 100 mM cysteamine (Sigma M-9768). Maturation Medium 3: TC199 (Gibco 11150-059), 10% FBS (Hyclone A-111D), 10 mg/ml ovine FSH (Sigma L-8174), 10 mg/ml ovine LH (Sigma L-5269), 1 mg/ml Estradiol (Sigma E-2257) and 100 mM cysteamine (Sigma M-9768).

Oocyte Enucleation

Typically, oocytes were stripped of cumulus cells after 17 hours in maturation medium by vortexing in 0.5 ml of TL-HEPES. The chromatin was stained with Hoechst 33342 (5 mg/ml, Sigma) in TL-HEPES solution for 15 minutes. Oocytes were then enucleated in TL-HEPES with or without calcium.

Nuclear Transfer

All nuclear transfers were performed in TL HEPES containing calcium regardless of what enucleation medium was used. Fusion was performed 19 hours after initiation of maturation using Sorbitol fusion medium with calcium (0.25 M sorbitol, 0.1 mM calcium acetate, 0.5 mM magnesium acetate and 1 mg/ml bovine serum albumin [Sigma #A7030]; pH 7.2) or without calcium (omit calcium acetate and increase magnesium acetate to 0.6 mM) and the following parameters: one 90V pulse for 30 used (GenAust Fusion Machine, Bracchus Marsh, Australia). After fusion, NTs were placed in CR2 medium with 3 mg/ml BSA until activation.

Oocyte Activation

Activation was performed approximately 24 hours after initiation of maturation by incubating the nuclear transfer embryos (NTs) with 10 μM ionomycin (calcium salt, Calbiochem #407952) in 3 ml of TL HEPES for 4 minutes followed by a TL HEPES rinse and a subsequent incubation in 1.9 mM 6-dimethylaminopurine (DMAP) (Sigma #D2629) for approximately 4 hours. The NTs were cultured in CR2 culture medium with 3 mg/ml BSA for 5-6 days.

When calcium-free enucleation and fusion solutions were used, the timing of fusion and activation were typically delayed to approximately 22 hours and 26 hours after initiation of maturation, respectively.

Embryo Manipulation

On day 5 or 6, cleaved NTs were moved into CR2 containing 15% charcoal stripped FBS (Hyclone cat. #SH30068.02). On day 7, blastocysts were loaded into embryo transfer straws for embryo transfer.

Embryo Transfer

A recipient ewe was selected from the recipient flock based on observed estrus behavior and was not allowed to consume feed for 24 hours prior to surgery. The ewe was anesthetized by intramuscular injection of xylazine (5 mg, Bayer Animal Health) and ketamine (400-500 mg, Fort Dodge Animal Health) and was placed in dorsal recumbancy in a surgical cradle. The wool was closely clipped from her caudal ventral abdomen and the surgical site was prepared by gently scrubbing the skin with Betadine soaked sponge gauze followed by rinsing with alcohol soaked sponge gauze. Lidocaine (60 mg) was injected under the skin on the midline 6 cm cranial to the mammary glands. A sterile drape was placed over the surgical site and a 4-6 cm incision was made through the skin and body wall on the midline just cranial to the mammary glands. Significant blood vessels were ligated or occluded with hemostats. Embryos were transferred into the uterine horn ipsilateral to the ovary with corpora lutei by puncturing the uterus near the utero-tubal juncture with the blunt end of a small suture needle and threading a 5.5-inch TomCat® catheter (Sherwood Medical) containing the embryos (1-4) into the uterine horn. After delivering the embryos into the uterus, the TomCat catheter was removed and the uterine horn was rinsed with sterile saline solution before relocation into the body cavity. Intramuscular injections of procaine penicillin G (3×10⁶ U, US Veterinary) and flunixin meglumine (100 mg, Schering-Plough) (an analgesic) were given post-surgically.

### Example 5

### Statin Treatment of Cultured Cells

Ovine Results

A lovastatin (A. G. Scientific, Inc. catalog #L-1043; M.W. 404.5) stock solution (100×; 10.115 mg lovastatin dissolved in 50 ml of 60% ethanol in water (v/v)) was diluted 1:100 in cell culture medium to obtain a final lovastatin concentration of 5 μM. Cells were cultured in this medium for 24 hours in a 5-10% C02, humidified air atmosphere at 37° C.

Thereafter, cells were treated in one of three different ways: 1) cells were washed twice with culture medium to remove lovastatin and then prepare cells for nuclear transfer as usual; (2) cells were washed twice with culture medium to remove lovastatin and then incubated in medium without lovastatin for 3 hours prior to preparation of the cells for nuclear transfer; or (3) cells were used in nuclear transfer without removal of lovastatin.

Ear cells derived from a 10-year-old crossbred blackface ewe were cultured in high glucose DMEM (Gibco cat. #10569-010) supplemented with 10% fetal bovine serum (Hyclone cat. #SH30070.03) and 0.1 mM 2-mercaptoethanol (Gibco cat. #21985-023). The ear cells (SA01-FB) were treated with lovastatin for 24 hours without a subsequent lovastatin-free incubation. On two different days, lovastatin treated cells were used in nuclear transfer to produce one and four blastocysts (day 7), respectively (see Table 1). The single blastocyst from one day of NT and the four blastocysts from the other day of NT were surgically transferred into estrus synchronized ewes (Tables 1 & 2). The ewe with 4 NT blastocysts became pregnant and gave birth to a healthy lamb.

Untreated SA01-FB cells were used to produce NT blastocysts that were transferred into 12 recipients (Table 1). Two of these recipients became pregnant but subsequently aborted (Table 2).

TABLE 1

| # of | Polar Body | Mat. time | # NTs | Time at | Number | # of | # emb. | |
|---|---|---|---|---|---|---|---|---|
| oocytes | formation | at fusion | fused | Act. | cleaved | Bl. | transferred | Status |
| 317 | | 19 hr | 124 | 24 hr | 48/118 (41%) | 2 | 2 | open |
| 242 | | 19 hr | 70 | 24 hr | 14/70 (20%) | 1 | 1 | Preg/ |
| | | | | | | | | Abort |
| 329 | 96/114 (67%) | 19.5 hr | 83 | 23.5 hr | 60/81 (74%) | 2 | 2 | open |
| 123 | | 19 hr | 52 | | 39/51 (76%) | 3 | 3 | open |
| 354 | 160/225 (71%) | 19 hr | 128 | 24 hr | 91/126 (72%) | 7 | 5 | open |
| 173 | 63/81 (77%) | 19 hr | 80 cycling | 24 hr | 74/80 (92.5%) | 1 | 3 (2 from | open |
| | | | cells | | | | 2/13) | |
| 288 | 52/67 (78%) | 19 hr | 103 (L0 cells) | 24 hr | 11/103 (10.7%) | 1 | 1 | open |
| 347 | 71/113 (63%) | 19 hr | 97 (L0 cells) | 24 hr | 68/97 (70%) | 4 | 4 | Lambed |
| | | | | | | | | Sep. 22, 2001 |
| 152 | 79/104 (76%) | 19 hr | 32 (Ca fusion) | 24 hr | 24/32 (75%) | 1 | 1 | open |
| 277 | 66/125 (53%) | 19 hr | 47 (Ca fusion) | 24.5 hr | 32/47 (68%) | 3 | 3 | open |
| | | | 49 (Ca free | | 28/49 (57%) | 1 | 1 | |
| | | | fus) | | | | | |
| 175 | | 19 hr | 40 (Ca fusion) | 24 hr | 20/40 (50%) | 2 | 1 | open |
| | | | 44 (Ca free | | 24/44 (55%) | 2 | 2 | |
| | | | fus) | | | | | |
| 155 | 32/52 (62%) | 19 hr | 71 (Ca free | 24 hr | 51/71 (72%) | 2 | 3 (1 from | open |
| | | | fus) | | | | 3/2) | |
| 252 | | 19 hr | 51 | 24 hr | 30/51 (59%) | 1 | 1 | open |
| 177 | 44/74 (59%) | 19 hr | 35 (Ca fusion) | 24 hr | 32/35 (91%) | 2 | 4 | Preg/ |
| | | | | | | | | Abort |
| | | | 35 (Ca free | | 28/35 (80%) | 2 | | |
| | | | fus) | | | | | |

TABLE 2
SHEEP PREGNANCY DATA Aug. 6, 2001

| | | Pregnancy | Pregnancy | Pregnancy |
|---|---|---|---|---|
| | | One | Two | Three |
| | Pregnancy Type | NT | NT | NT |
| | Cell line | SA01-FB00 | SA01-FB00 | SA01-FB00 |
| | | | Lovastatin treated | |
| CELLS | Media | hDMEM | hDMEM | hDMEM |
| | Fresh/Frozen cells | fresh | frozen | frozen |
| | Sex of Fetus | Female | Female | Female |
| | Age in culture at NT | 45 days | 28 days | 51 days |
| | Number of Passages | 5 | 3 | 6 |
| | Age @ stripping | 17 hours | 17 hours | 17 hours |
| NT | Age and number @ fusion | 19 hours | 19 hours | 19 hours |
| | Age and number @ | 24 hours | 24 hours | 24 hours |
| | activation | | | |
| | Activation Protocol | 2 × iono/DMAP | 2 × iono/DMAP | 2 × iono/DMAP |
| | Time in DMAP | 4 hours | 4¼ hours | 4¼ hours |
| | Transfer date | Feb. 10, 2001 | Feb. 28, 2001 | Mar. 23, 2001 |
| RECIPIENT | Number transferred | 1 Day 7 | 4 Day 7 | 2 +Ca, 2 −Ca |
| | & stage | Blastocyst | Blastocyst | Blastocyst |
| | Farrowed/Aborted | abort | farrowed | abort |
| | Recipient #, (synchrony) | #34, −12 hr | #56, −12 hr | #68, −12 hr |

Bovine Results

Similar results were obtained using statin-treated bovine cells in nuclear transfer procedures. Ear cells derived from a newborn cloned calf were cultured in α-MEM (Gibco cat. #32561-037) supplemented with 10% fetal bovine serum (Hyclone cat. #SH30070.03) and 0.1 mM 2-mercaptoethanol (Gibco cat. #21985-023). The ear cells were treated with lovastatin for 24 hours without a subsequent lovastatin-free incubation. On two different days, lovastatin treated cells were used in nuclear transfer to produce 6 blastocysts, respectively, by day 7 of culture (19% development to blastocyst). One or two blastocysts were non-surgically transferred into 5 recipients of which two became pregnant (Table 3). One pregnancy aborted by day 32 while the other pregnancy produced a live calf.

TABLE 3

| | | | Activation | | | Emb. Devel. (% |
|---|---|---|---|---|---|---|
| RecipID | TXStatus | CellLineID | Date | Sex | Notes | Blastocyst) |
| 1505 | Abort | C188/Firstdown-FB000 | Feb. 22, 2001 | M | LOVASTATIN | 19% |
| 1350 | Open | C188/Firstdown-FB000 | Mar. 1, 2001 | M | LOVASTATIN | 19% |
| 1491A | Open | C188/Firstdown-FB000 | Feb. 22, 2001 | M | LOVASTATIN | 19% |
| 1510 | Open | C188/Firstdown-FB000 | Feb. 22, 2001 | M | LOVASTATIN | 19% |
| 1579 | Calved | C188/Firstdown-FB000 | Mar. 1, 2001 | M | LOVASTATIN | 19% |

One skilled in the art readily appreciates that the present invention is well adapted to carry out the objects and obtain the ends and advantages mentioned, as well as those inherent therein. The cell lines, embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein where any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are set forth within the following claims.

What is claimed is:

1. A method for replicating an immune response to at least one antigen of interest in a first non-human mammal in a second non-human mammal, comprising:
providing a second mammal prepared by nuclear transfer cloning using said first mammal as a founder mammal; and
producing an immune response to said antigen of interest in the second mammal that is substantially identical to the immune response to said antigen of interest in said first mammal.

2. The method of claim 1, wherein the step of producing an immune response in the second mammal comprises subjecting the second mammal to said antigen of interest under conditions that stimulate the immune system of the second mammal to produce an immune response that is substantially identical to the immune response to said antigen of interest in said first mammal.

3. The method of claim 2, wherein the step of subjecting the second mammal to said antigen of interest under conditions that stimulate the immune system comprises immunizing the second mammal with said antigen of interest.

4. The method of claim 1, wherein the first mammal is selected from the group consisting of sheep, cows, pigs, and goats.

5. The method of claim 1, wherein the replicated immune response comprises one or more antibodies that recognize said antigen of interest.

6. The method of claim 5, wherein said one or more antibodies comprise a polyclonal antibody.

7. The method of claim 5, further comprising isolating one or more of said antibodies that recognize said antigen of interest.

8. The method of claim 1, wherein the step of producing an immune response to said antigen of interest in the second mammal comprises adoptively transferring one or more cells of the immune system obtained from the first mammal to the second mammal.

9. The method of claim 8, wherein the step of adoptively transferring one or more cells comprises obtaining one or more cells of the immune system of the first mammal, and transferring said one or more immune system cells to the second mammal.

10. The method of claim 9, further comprising selecting one or more immune system cells responsible for an immune response to said antigen of interest, and transferring the selected immune system cells to the second mammal.

11. The method of claim 9, wherein the step of adoptively transferring one or more cells further comprises increasing the number of the immune system cells obtained from said first mammal prior to transfer of said immune system cells to the second mammal.

12. The method of claim 9, wherein the immune system cells obtained from said first mammal comprise T-lymphocytes and B-lymphocytes.

13. The method of claim 9, wherein the immune system cells obtained from said first mammal comprise memory cells and antibody secreting cells.

14. The method of claim 9, wherein the immune system cells are obtained from a source selected from the group consisting of one or more lymph nodes of the first mammal, the bone marrow if the first mammal, and the peripheral blood of the first mammal.

15. The method of claim 8, wherein the immune system cells are obtained from one or more lymph nodes of the first mammal.

16. The method of claim 8, wherein the immune system of the second mammal is at least partially ablated prior to the step of adoptively transferring one or more immune system cells.

17. The method of claim 16, wherein the immune system of the second mammal is substantially fully ablated prior to the step of adoptively transferring one or more immune system cells.

18. The method of claim 8, further comprising immunizing the second mammal with said antigen of interest following the step of adoptively transferring one or more immune system cells.

19. A non-human mammal prepared by nuclear transfer cloning, comprising an immune system that provides an immune response to at least one antigen of interest that is substantially the same as the immune response to said at least one antigen of interest in a founder mammal used to establish said mammal.

20. The mammal of claim 19, wherein the founder mammal is selected from the group consisting of sheep, cows, pigs, and goats.

21. The mammal of claim 19 wherein the immune response to said antigen of interest comprise antibodies that recognize said antigen of interest.

22. The mammal of claim 21, wherein the antibodies comprise polyclonal antibodies.

23. A method of producing a mammalian nuclear transfer embryo, comprising:
contacting a mammalian cell with a compound that is an inhibitor of cholesterol biosynthesis; and
using said mammalian cell, or a nucleus thereof, in a nuclear transfer procedure to produce said nuclear transfer embryo.

24. A method of producing a mammalian nuclear transfer embryo, comprising:
contacting a mammalian cell with an inhibitor of hydroxymethylglutaryl-CoA reductase, or a salt, ester, or lactone thereof; and
using said mammalian cell, or a nucleus thereof, in a nuclear transfer procedure to produce said nuclear transfer embryo.

25. The method of claim 23 wherein said nuclear transfer procedure comprises:
(a) translocating said mammalian cell, or a nucleus thereof, into an enucleated recipient cell of the same species as the mammalian cell to form a hybrid cell; and
(b) activating said hybrid cell to provide said nuclear transfer embryo.

26. The method of claim 23, wherein said mammal is an ungulate.

27. The method of claim 26, wherein said ungulate is a bovine, porcine, or ovine.

28. The method of claim 23, wherein the mammalian cell is a cultured cell.

29. The method of claim 23, wherein the inhibitor of cholesterol biosynthesis is selected from the group consisting of lovastatin, simvistatin, pravastatin, fluvastatin, atorvastatin, and cerivastatin, or a salt, ester, or lactone thereof.

30. The method of claim 23, wherein the inhibitor of hydroxymethylglutaryl-CoA reductase is selected from the group consisting of lovastatin, simvistatin, pravastatin, fluvastatin, atorvastatin, and cerivastatin, or a salt, ester, or lactone thereof.

31. The method of claim 23, wherein the mammalian cell is obtained by culturing one or more cells taken from a live-born mammal.

32. The method of claim 23, wherein the mammalian cell is obtained by culturing one or more cells taken from a fetal mammal.

33. The method of claim 23, wherein the mammalian cell is obtained by culturing one or more cells taken from a mammalian embryo.

34. The method of claim 23, wherein the mammalian cell is a transgenic cell.

35. A method of producing a mammalian fetus, comprising:
transferring said nuclear transfer embryo of claim 23 into a maternal animal of the same species as the mammalian cell so as to develop into said mammalian fetus.

36. A method of producing a mammalian animal, comprising:
transferring said nuclear transfer embryo of claim 23 into a maternal animal of the same species as the mammalian cell so as to develop into said mammalian fetus that undergoes parturition to produce said mammalian animal.
